SEQUENCE LISTING (12 sequences)

SEQ ID NO: 1 - DNA, 15277 bp, Human Respiratory Syncytial Virus
acgcgaaaaaatgcgtacaacaaacttgcgtaaaccaaaaaaatggggcaaataagaatttgataagtaccacttaaatttaactcctttggttagagatgggcagcaactcattgagtatgataaaagttagattgcaaaatctgtttgacaatgatgaagtagcattgttaaaaataacatgctatactgacaaattaatacagttaactaatgctttggctaaggcagttatacatacaatcaaattgaatggcattgtatttgtgcatgttattacaagtagtgatatttgccctaataataatattgtagtgaaatccaatttcacaacaatgccagtattacaaaatggaggttatatatgggaaatgatggaattaacacactgctctcaacctaatggcctaatagatgacaattgtgaaattaaattctccaaaaaactaagtgattcaacaatgaccaattatatgaatcaattatctgaattacttggatttgacctcaatccataaatcataataaatatcaactagcaaatcaatgtcactaacaccattagttaatataaaacttgacagaagataaaaatggggcaaataaatcgattcagccgacccaaccatggacacaacacacaatgataccacaccacaaagactgatgatcacagacatgagaccattatcgcttgagactataataacatctctaaccagagatatcataacacataaatttatatacttgataaatcatgaatgcatagtgagaaaacttgatgaaagacaggccacatttacgtttctggtcaactatgaaatgaaactattgcacaaagtgggaagcactaaatataaaaaatatactgaatacaacacaaaatatggcactttccctatgccaatatttatcaatcatgatgggttcttagaatgcattggcattaagcctaccaagcacacacccataatatacaagtatgatctcaatccatgaatatcaaaccaagattcaaacaatccgaaataacaactttatgcataatcacactccatagtccagatggagcctgaaaattatagttatttaaaattaaggagagacataagatgaaagatggggcaaatacaaaaatggctcctagcaaagtcaagttgaatgatacactcaacaaagatcaacctccatcatccagcaaatataccatccaacggagcacaggagacagcaccgacactcccaattatgatgtgcagaaacacaccaataagctatgtggcatgttattaatcacagaagatgctaatcataaattcactgggttaataggtatgttatatgctatgtctagattaggaagagaagacaccataaaaatactcaaagatgcgggatatcatgttaaggcaaatggagtggatgtaacaacacatcgtcaagacattaatgggaaagaaatgaaatttgaagtgttaacattagcaagcttaacaactgaaattcaaatcaacattgagatagaatctagaaaatcctacaaaaaaatgctaaaagaaatgggagaggtggctccagaatacaggcatgactctcctgattgtgggatgataatattatgtatagcagcattagtaataaccaaattagcagcaggagatagatcaggtcttacagctgtgattaggagagctaataatgtcctaaaaaatgaaatgaaacgttataaaggtttattacccaaggatatagccaacagcttctatgaagtgtttgaaaaatatcctcactttatagatgtttttgttcattttggtatagcacaatcttctaccagaggtggcagtagagttgaagggatttttgcaggattgtttatgaatgcctatggtgcagggcaagtgatgttacggtggggggtcttagcaaaatcagttaaaaacattatgttaggacacgctagtgtacaagcagaaatggaacaagttgtggaggtgtatgagtatgctcagaaattgggtggagaagcaggattctaccatatattgaacaacccaaaagcatcactattatctttgactcaatttcctcacttctctagtgtagtattgggcaatgctgctggcctaggcataatgggagaatacagaggtacaccaaggaatcaagatttatatgatgctgcaaaagcatatgctgaacaactcaaagaaaatggtgtgattaactacagtgtattagatttgacagcagaagaactagaggctatcaaacatcagcttaatccaaaagataatgatgtagagctttgagttaataaaaaggtggggcaaataaatcatcatggaaaagtttgctcctgaattccatggagaagatgcaaacaacagagccaccaaattcctagaatcaataaagggcaaattcacatcacccaaagatcccaagaaaaaagatagtatcatatctgtcaactcaatagatatagaagtaaccaaagaaagccctataacatcaaattcaaccattataaacccaataaatgagacagatgatactgtagggaacaagcccaattatcaaagaaagcctctagtaagtttcaaagaagaccctacgccaagtgataatcctttttcaaaactatacaaagaaaccatagaaacatttgataacaatgaagaagaatctagctattcatatgaagaaataaatgatcagacaaacgataatataacagcaagattagataggattgatgagaaattaagtgaaatactaggaatgcttcacacattagtagtagcgagtgcaggacccacatctgctcgggatggtataagagatgccatggttggtttaagagaagaaatgatagaaaaaatcagaactgaagcattaatgaccaatgacagactagaagctatggcaagactcaggaatgaagaaagtgaaaagatggcaaaagacacatcagatgaagtgtctctcaatccaacatcagagaaactgaacaacctgttggaagggaatgatagtgacaatgatctatcacttgaagatttctgattagctaccaaactgtacatcaaaacacaacaccaatagaaaaccaacaaacaaaccaactcacccatccaaccaaacatctatctgctgattagccaaccagccaaaaaacaaccagccaatctaaaactagccacccggaaaaaatcgatactatagttacaaaaaaagatggggcaaatatggaaacatacgtgaataaacttcacgagggctccacatacacagctgctgttcaatacaatgtcctagaaaaagacgatgatcctgcatcacttacaatatgggtgcccatgttccaatcatccatgccagcagatctactcataaaagaactagccaatgtcaatatactagtgaaacaaatatccacacccaagggaccctcattaagagtcatgataaactcaagaagtgcagtgctagcacaaatgcccagcaaatttaccatatgtgccaatgtgtccttggatgaaagaagcaagctggcatatgatgtaaccacaccctgtgaaattaaggcatgcagtctaacatgcctaaaatcaaaaaatatgttaactacagttaaagatctcactatgaaaacactcaacccaacacatgacatcattgctttatgtgaatttgaaaatatagtaacatcaaaaaaagtcataataccaacatacctaagatctatcagcgtcagaaataaagatctgaacacacttgaaaatataacaaccactgaattcaaaaatgccattacaaatgcaaaaatcatcccttactcaggattactgttagtcatcacagtgactgacaacaaaggagcattcaaatacataaagccacaaagtcaattcatagtagatcttggagcttacctagaaaaagaaagtatatattatgttacaacaaattggaagcacacagctacacgatttgcaatcaaacccatggaagattaacctttttcctctacattaatgagtagattcatacaaactttctaactacattcttcacttcacaatcataatcaccaaccctctgtggttcaatcaatcaaacaaaactcatcaggagttccagatcatcccaagtcattgttcatcagatccagtactcaaataagttaataaaaaatccacatggggcaaataatcattgagggtaatccaactaatcacaacatctgtcaacatagacaagtcaacacgctagataaaatcaaccaatggaaaatacatccataactatagaattctcaagcaaattctggccttactttacactaatacacatgataacaacaataatctctttgataatcataatctccatcatgattgcaatactaaacaaactctgcgaatataatgtattccataacaaaacctttgagctaccaagagctcgagtcaatacatagcatttaccaatctgatagctcaaaacagtaaccttgcatttgtaaatgaactaccctcacttcttcacaaaaccacatcaacatctcaccatgcaagccatcatctataccataaagtagttaattaaaaaatagtcataacaatgaactaggatattaagaccaaaaacaacgctggggcaaatgcaaacatgtccaaaaccaaggaccaacgcaccgccaagacactagaaaggacctgggacactctcaatcatctattattcatatcatcgtgcttatacaagttaaatcttaaatctatagcacaaatcacattatctattttggcaatgataatctcaacctcacttataattgcagccatcatattcatagcctcggcaaaccacaaagtcacactaacaactgcaatcatacaagatgcaacgaaccagatcaagaacacaaccccaacatacctcactcagaatccccagcttggaatcagcttctccaatctgtccggaactacatcacaatccaccaccatactagcttcaacaacaccaagtgctgagtcaaccccacaatccacaacagtcaagatcaaaaacacaacaacaacccaaatattacctagcaaacccaccacaaaacaacgccaaaataaaccacaaaacaaacccaacaatgattttcactttgaagtgttcaattttgtaccctgcagcatatgcagcaacaatccaacctgctgggccatctgcaagagaataccaaacaaaaaacctggaaagaaaaccaccaccaagcccacaaaaaaaccaaccctcaagacaaccaaaaaagatcccaaacctcaaaccacaaaaccaaaggaagtactcactaccaagcctacaggaaagccaaccatcaacaccactaaaacaaacatcagaactacactgctcacctccaacaccaaaggaaatccagaacacacaagtcaagaggaaaccctccactcaaccacctccgaaggctatctaagcccatcccaagtctatacaacatccggtcaagaggaaaccctccactcaaccacctccgaaggctatctaagcccatcacaagtctatacaacatccgagtacctatcacaatctctatcttcatccaacacaacaaaatgatagtcattaaaaagcgtattgttgcaaaaagccatgaccaaatcaaacagaatcaaaatcaactctggggcaaataacaatggagttgccaatcctcaaaacaaatgctattaccacaatccttgctgcagtcacactctgtttcgcttccagtcaaaacatcactgaagaattttatcaatcaacatgcagtgcagttagcaaaggctatcttagtgctctaagaactggttggtatactagtgttataactatagaattaagtaatatcaaggaaaataagtgtaatggtacagacgctaaggtaaaattaataaaacaagaattagataaatataaaaatgctgtaacagaattgcagttgctcatgcaaagcacaccagcagccaacagtcgagccagaagagaactaccaagatttatgaattatacactcaacaataccaaaaacaccaatgtaacattaagtaagaaaaggaaaagaagatttcttggatttttgttaggtgttggatctgcaatcgccagtggcattgccgtatccaaggtcctgcacctagaaggggaagtgaacaaaatcaaaagtgctctactatccacaaacaaggctgtagtcagcttatctaatggagtcagtgtcttaaccagcaaggtgttagacctcaaaaactatatagataaacagttgttacctattgttaacaagcaaagctgcagcatatcaaacattgaaactgtgatagagttccaacaaaagaacaacagactactagagattaccagagaatttagtgttaatgcaggtgtaactacacctgtaagcacttatatgttaactaatagtgagttattatcattaatcaatgatatgcctataacaaatgatcagaaaaagttaatgtccagcaatgttcaaatagttagacagcaaagttactctatcatgtcaataataaaagaggaagtcttagcatatgtagtacaattaccactatatggtgtaatagatactccttgttggaaactacacacatctcctctatgtacaaccaacacaaaggaaggatccaacatctgcttaacaagaaccgacagaggatggtactgtgacaatgcaggatcagtatcctttttcccacaagctgaaacatgtaaagttcaatcgaatcgggtgttttgtgacacaatgaacagtttaacattaccaagtgaggtaaatctctgcaacattgacatattcaaccccaaatatgattgcaaaattatgacttcaaaaacagatgtaagcagctccgttatcacatctctaggagccattgtgtcatgctatggcaaaaccaaatgtacagcatccaataaaaatcgtgggatcataaagacattctctaacgggtgtgattatgtatcaaataagggggtggatactgtgtctgtaggtaatacattatattatgtaaataagcaagaaggcaaaagtctctatgtaaaaggtgaaccaataataaatttctatgatccattagtgttcccctctgatgaatttgatgcatcaatatctcaagtcaatgagaaaattaatcagagtctagcatttatccgtaaatcagatgaattattacataatgtaaatgctggtaaatccaccacaaatatcatgataactaccataattatagtaattatagtaatattgttagcattaattgcagttggactgcttctatactgcaaggccagaagcacaccagtcacattaggtaaggatcaactgagtggtataaataatattgcatttaataactgaataaaaatagcacctaatcatattcttacaatggttcgctatttgaccatagataacccatctatcattagattatcctaaaatttgaacttcatcacaactttcatctataaaccatctcacttacactttttaagtagattcctattttatagttatataaaacaattgaataccaaattaacttactatttgtaaaaatgagaactggggcaaatatgtcacgaaggaatccttgcaaattcgaaattcgaggtcattgcttgaatggtaaaaggtgtcattttagtcataattattttgaatggccaccccatgcactgcttgtaagacaaaactttatgttaaacagaatacttaagtctatggataaaagcatagatactttgtcagaaataagtggagctgcagagttggacagaacagaagagtatgccctcggtgtagttggagtgctagagagttatataggatcaataaataatataactaaacaatcagcatgtgttgccatgagcaaactccttactgaactcaacagcgatgacatcaaaaaactaagggacaatgaagagccaaactcacccaaagtaagagtgtacaatactgtcatatcatatattgaaagcaacaggaagaacaataaacaaactatccatctgttaaaaagattgccagcagacgtattgaagaaaaccatcaaaaacacattggatatccacaagagcataaccatcaataacccaaaagaatcaactgttagtgatacgaacgaccatgccaaaaataatgatactacctgacaaatatccttgtagtataaattccatactaataacaagtaattgtagagtcactatgtataatcaaaaaaacacactatatatcaatcaaaacaaccaaaataaccatatatacccaccggatcaaccattcaatgaaatccattggacctctcaagacttgattgatgcaactcaaaattttctacaacatctaggtattactgatgatatatacacaatatatatattagtgtcataatactcaatcctaatacttaccacatcatcaaattattaactcaaacaattcaagctatgggacaaaatggatcccattattagtggaaattctgctaatgtttatctaactgatagttatttaaaaggtgttatttctttctcagaatgtaacgctttaggaagttacatattcaatggtccttatctcaaaaatgattataccaacttaattagtagacaaaatccattaatagaacacataaatctaaagaaactaaatataacacagtccttaatatctaagtatcataaaggtgaaataaaaatagaagaacctacttactttcagtcattacttatgacatacaagagtatgacctcgtcagaacagactactactactaatttacttaaaaagataataagaagagctatagaaatcagtgatgtcaaagtctatgctatattgaataaactggggctcaaagaaaaagacaagattaaatccaataatggacaagatgaagacaactcagtcattactaccataatcaaagatgatatacttttagctgtcaaggataatcaatctcatcttaaagcagacaaaaatcaatccacaaaacaaaaagatacaatcaaaacaacacttttgaagaaattaatgtgttcaatgcaacatcctccatcatggttaatacattggtttaatttatacacaaaattaaacagcatattaacacaatatcgatctagtgaggtaaaaaaccatggttttatattgatagataatcatactcttagtggattccaatttattttgaatcaatatggttgtatagtttatcataaggaactcaaaagaattactgtgacaacttataatcaattcttgacatggaaagatattagccttagtagattaaatgtttgtttgattacatggattagtaactgtttgaacacattaaacaaaagcttaggcttaagatgtggattcaataatgttatcttgacacaattattcctttatggagattgtatactaaaactattccacaatgaggggttctacataataaaagagatagagggatttattatgtctctaattttaaatataacagaagaagatcaattcagaaaacggttttataatagtatgctcaacaacatcacagatgccgccaacaaagctcaaaaaaatctgctatcaagagtatgtcatacattattagataagacaatatcagataatataataaatggcagatggataattctattgagcaagttcctaaaattaattaagcttgcaggtgacaataacctcaacaatctgagtgaattatattttttgttcaggatatttggacacccaatggtagatgaaagacaagccatggatgctgttaaagttaattgcaacgagaccaaattttacttgttaagtagtttgagtatgttaagaggagcttttatatatagaattataaaagggtttgtaaataattacaacagatggcctactttaagaaatgccattgtcttacccttaagatggttaacttactataaactaaacacttatccttccttgttggaacttacagaaagagatttgattgttctatcaggactacgtttctatcgagagtttcggttgcctaaaaaagtggatcttgaaatgatcataaatgataaggctatatcacctcctaaaaatttaatatggactagtttccctagaaattatatgccgtcacacatacaaaattatatagaacatgaaaaattaaaattctctgatagtgataaatcaagaagagtattagagtattatttaagagataacaaattcaatgaatgtgatttacacaactgtgtagttaatcaaagttatcttaacaacccgaatcatgtggtatcattgacaggcaaagaaagagaactcagtgtaggtagaatgtttgcaatgcaaccaggaatgttcagacaagttcaaatattagcagagaaaatgatagcagaaaacatattacaatttttccctgaaagtcttacaagatatggtgatctagaactacagaaaatattagaattgaaagcaggaataagtaacaaatcaaatcgttacaatgataattacaacaattacattagtaagtgctctatcatcacagatctcagcaaattcaatcaagcatttcgatatgaaacatcatgtatttgtagtgatgtactggatgaactgcatggtgtacaatctctattttcctggttacatttaactattcctcatgtcacaataatatgcacatataggcatgcacccccctatataaaggatcatattgtagatcttaacaatgtagatgagcaaagtggactatatagatatcatatgggtggtatcgaagggtggtgtcaaaaactatggactatagaagctatatcactattagatctaatatctctcaaagggaaattctcaattactgctttaattaatggtgacaatcaatcaatagatataagtaaaccagtcagactcatggaaggtcaaactcatgctcaagcagattatttgctagcattaaatagtctcaaattactgtataaagagtatgcaggaataggccacaaattaaaaggaactgagacttatatatcgagagatatgcaatttatgagtaaaacgatccaacataacggtgtatattacccagctagtataaagaaagtcctaagagtgggaccgtggataaacactatacttgatgacttcaaagtgagtctagaatctataggtagtttgacacaagaattagaatatagaggtgaaagtctattatgcagtttaatatttaggaatgtatggttatataatcaaattgcattacaacttaaaaatcatgcattatgtaacaacaaattatatttggatatattaaaagttctaaaacacttaaaaaccttttttaatcttgataacattgatacagcattaacattgtatatgaatttgcccatgttatttggtggtggtgatcccaacttgttatatcgaagtttctatagaagaactcctgatttcctcacagaggctatagttcactctgtgttcatacttagttattatacaaaccatgatttaaaagataaacttcaagatctgtcagatgatagattgaataagttcttaacatgcataatcacgtttgacaaaaaccccaatgctgaattcgttacattgatgagagatcctcaagctttaggatctgagaggcaagctaaaattactagcgaaatcaatagactggcagttaccgaggttttgagcacagctccaaacaaaatattttccaaaagtgcacaacactataccactacagagatagatcttaatgatattatgcaaaatatagaacctacatatcctcacgggttaagagttgtttatgaaagtttacccttttataaagcagagaaaatagtaaatcttatatccggtacaaaatctataactaacatactggaaaagacttctgccatagacttaacagatattgatagagccactgagatgatgaggaaaaacataactttgcttataaggatattaccattagattgtaacagagataaaagagaaatattgagtatggaaaacctaagtattactgaattaagcaaatacgttagagaaagatcttggtctttatccaatatagttggtgttacatcacccagtatcatgtatacaatggacataaaatatacaacaagcactatagctagtggcataatcatagagaaatataatgtcaacagtttaacacgtggtgagagaggacccactaaaccatgggttggttcatctacacaagagaaaaagacaatgccagtttataatagacaagttttaaccaaaaaacagagagatcaaatagatctattagcaaaattggattgggtgtatgcatctatagataacaaggatgaatttatggaggaacttagcataggaactcttgggttaacatatgagaaggccaaaaaattattcccacaatatttaagtgttaactatttgcatcgtcttacagtcagtagtagaccatgtgaattccctgcatctataccagcttatagaactacaaattatcactttgatactagccctattaatcgcatattaacagaaaagtatggtgatgaagatattgatatagtattccaaaactgtataagctttggccttagcttaatgtctgtagtagaacaatttactaatgtgtgtcctaacagaattattctcatacccaagcttaatgagatacatttgatgaaacctcccatattcacaggtgatgttgatattcacaagttaaaacaagtgatacaaaaacaacatatgtttttaccagacaaaataagtttgactcaatatgtggaattattcttaagtaataaaacactcaaatctggatctaatgttaattctaatttaatattggcgcataagatatctgactattttcataatacttacattttaagtactaatttagctggacattggattcttattatacaacttatgaaagattctaagggtatttttgaaaaagattggggagagggatatataactgatcatatgttcattaatttgaaagttttcttcaatgcttataagacatatctcttgtgttttcataaaggttacggcagagcaaagctggagtgtgatatgaatacttcagatctcctatgtgtattggaattaatagacagtagttattggaagtctatgtctaaggtgtttttagaacaaaaagttatcaaatacattcttagccaggatgcaagtttacatagagtaaaaggatgtcatagcttcaaactatggtttcttaaacgtcttaatgtagcagaattcacggtttgcccttgggttgttaacatagattatcatccaacacatatgaaagcaatattaacttatattgatcttgttagaatgggattgataaatatagatagaatatacattaaaaataaacacaagttcaatgatgagttttatacttctaatctgttttacattaattataacttctcagataatactcatctattaactaaacatataaggattgctaattccgaattagaaagtaattacaacaaattatatcatcccacaccagaaaccctagaaaatatactaaccaatccggttaaaagtaatggaaaaaagacactgagtgactattgtataggtaaaaatgttgactcaataatgttaccatcgttatctaataagaagcttattaaatcgtctacaatgattagaaccaattgcagcagacaagatttgtataatttatttcctacggttgtgattgataaaattatagatcattcaggyaatacagccaaatctaaccaactttacactactacttctcatcaaatatccttagtgcacaatagcacatcactttattgcatgcttccttggcatcatattaatagattcaattttgtatttagttctacaggttgtaaaattagtatagagtatattttaaaagatcttaaaattaaggatcctaattgtatagcattcataggtgaaggagcagggaatttattattgcgtacagtagtggaacttcatcctgatataagatatatttacagaagtctgaaagattgcaatgatcatagtttaccaattgagtttttaaggctgtacaatggacatatcaacattgattatggtgaaaatttgaccattcctgctacagatgcaaccaacaacattcattggtcttatttacatataaagtttgctgaacctatcagtctttttgtctgtgatgctgaattgcctgtaacagtcaactggagtaagattataatagagtggagcaagcatgtaagaaaatgcaagtactgttcttcagttaataaatgtacattaatagtaaaatatcatgctcaagatgatatcgatttcaaattagacaacataactatattaaaaacttatgtatgcttaggcagtaagttaaagggatctgaagtttacttagtccttacaataggtcctgcaaatgtgttcccagtatttaatgtagcacaaaatgctaaattgatactatcaagaaccaaaaatttcatcatgcctaaaaaagctgataaagagtctattgatgcaaatattaagagtttgataccctttctttgttaccctataacaaaaaaaggaattaataccgcattgtctaaattaaagagtgttgttagtggagatatactatcatattctatagctggacgtaatgaagttttcagcaataaacttataaatcataagcatatgaacatcttaaagtggttcaatcatgttttaaatttcagatcaacagaattaaactataatcatttatatatggtagaatctacttatcctcatctaagtgaattgttaaacagcttgactaccaatgaacttaaaaaactgattaaaatcacaggtagtttgttatacaacttttataatgaataatgagcaaaaatcttataacaaaaatagctacacactaacattgtattcaattatagttatttaaaattaataattatataatttttaataacttctagtgaactaatcctaaaattatcattttgatctaggaagaataagtttaaatccaaatctaattggtttatatgtatattaactaaattacgagatattagtttttgacactttttttctcgt

SEQ ID NO: 2 - protein, 139 aa, Human Respiratory Syncytial Virus
MGSNSLSMIKVRLQNLFDNDEVALLKITCYTDKLIQLTNALAKAVIHTIKLNGIVFVHVITSSDICPNNNIVVKSNFTTMPVLQNGGYIWEMMELTHCSQPNGLIDDNCEIKFSKKLSDSTMTNYMNQLSELLGFDLNP

SEQ ID NO: 3 - protein, 124 aa, Human Respiratory Syncytial Virus
MDTTHNDTTPQRLMITDMRPLSLETIITSLTRDIITHKFIYLINHECIVRKLDERQATFTFLVNYEMKLLHKVGSTKYKKYTEYNTKYGTFPMPIFINHDGFLECIGIKPTKHTPIIYKYDLNP

SEQ ID NO: 4 - protein, 391 aa, Human Respiratory Syncytial Virus
MAPSKVKLNDTLNKDQPPSSSKYTIQRSTGDSTDTPNYDVQKHTNKLCGMLLITEDANHKFTGLIGMLYAMSRLGREDTIKILKDAGYHVKANGVDVTTHRQDINGKEMKFEVLTLASLTTEIQINIEIESRKSYKKMLKEMGEVAPEYRHDSPDCGMIILCIAALVITKLAAGDRSGLTAVIRRANNVLKNEMKRYKGLLPKDIANSFYEVFEKYPHFIDVFVHFGIAQSSTRGGSRVEGIFAGLFMNAYGAGQVMLRWGVLAKSVKNIMLGHASVQAEMEQVVEVYEYAQKLGGEAGFYHILNNPKASLLSLTQFPHFSSVVLGNAAGLGIMGEYRGTPRNQDLYDAAKAYAEQLKENGVINYSVLDLTAEELEAIKHQLNPKDNDVEL

SEQ ID NO: 5 - protein, 241 aa, Human Respiratory Syncytial Virus
MEKFAPEFHGEDANNRATKFLESIKGKFTSPKDPKKKDSIISVNSIDIEVTKESPITSNSTIINPINETDDTVGNKPNYQRKPLVSFKEDPTPSDNPFSKLYKETIETFDNNEEESSYSYEEINDQTNDNITARLDRIDEKLSEILGMLHTLVVASAGPTSARDGIRDAMVGLREEMIEKIRTEALMTNDRLEAMARLRNEESEKMAKDTSDEVSLNPTSEKLNNLLEGNDSDNDLSLEDF

SEQ ID NO: 6 - protein, 256 aa, Human Respiratory Syncytial Virus
METYVNKLHEGSTYTAAVQYNVLEKDDDPASLTIWVPMFQSSMPADLLIKELANVNILVKQISTPKGPSLRVMINSRSAVLAQMPSKFTICANVSLDERSKLAYDVTTPCEIKACSLTCLKSKNMLTTVKDLTMKTLNPTHDIIALCEFENIVTSKKVIIPTYLRSISVRNKDLNTLENITTTEFKNAITNAKIIPYSGLLLVITVTDNKGAFKYIKPQSQFIVDLGAYLEKESIYYVTTNWKHTATRFAIKPMED

SEQ ID NO: 7 - protein, 64 aa, Human Respiratory Syncytial Virus
MENTSITIEFSSKFWPYFTLIHMITTIISLIIIISIMIAILNKLCEYNVFHNKTFELPRARVNT

SEQ ID NO: 8 - protein, 321 aa, Human Respiratory Syncytial Virus
MSKTKDQRTAKTLERTWDTLNHLLFISSCLYKLNLKSIAQITLSILAMIISTSLIIAAIIFIASANHKVTLTTAIIQDATNQIKNTTPTYLTQNPQLGISFSNLSGTTSQSTTILASTTPSAESTPQSTTVKIKNTTTTQILPSKPTTKQRQNKPQNKPNNDFHFEVFNFVPCSICSNNPTCWAICKRIPNKKPGKKTTTKPTKKPTLKTTKKDPKPQTTKPKEVLTTKPTGKPTINTTKTNIRTTLLTSNTKGNPEHTSQEETLHSTTSEGYLSPSQVYTTSGQEETLHSTTSEGYLSPSQVYTTSEYLSQSLSSSNTTK

SEQ ID NO: 9 - protein, 574 aa, Human Respiratory Syncytial Virus
MELPILKTNAITTILAAVTLCFASSQNITEEFYQSTCSAVSKGYLSALRTGWYTSVITIELSNIKENKCNGTDAKVKLIKQELDKYKNAVTELQLLMQSTPAANSRARRELPRFMNYTLNNTKNTNVTLSKKRKRRFLGFLLGVGSAIASGIAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSSNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNIDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLALIAVGLLLYCKARSTPVTLGKDQLSGINNIAFNN

SEQ ID NO: 10 - protein, 194 aa, Human Respiratory Syncytial Virus
MSRRNPCKFEIRGHCLNGKRCHFSHNYFEWPPHALLVRQNFMLNRILKSMDKSIDTLSEISGAAELDRTEEYALGVVGVLESYIGSINNITKQSACVAMSKLLTELNSDDIKKLRDNEEPNSPKVRVYNTVISYIESNRKNNKQTIHLLKRLPADVLKKTIKNTLDIHKSITINNPKESTVSDTNDHAKNNDTT

SEQ ID NO: 11 - protein, 88 aa, Human Respiratory Syncytial Virus
MPKIMILPDKYPCSINSILITSNCRVTMYNQKNTLYINQNNQNNHIYPPDQPFNEIHWTSQDLIDATQNFLQHLGITDDIYTIYILVS

SEQ ID NO: 12 - protein, 2165 aa, Human Respiratory Syncytial Virus
MDPIISGNSANVYLTDSYLKGVISFSECNALGSYIFNGPYLKNDYTNLISRQNPLIEHINLKKLNITQSLISKYHKGEIKIEEPTYFQSLLMTYKSMTSSEQTTTTNLLKKIIRRAIEISDVKVYAILNKLGLKEKDKIKSNNGQDEDNSVITTIIKDDILLAVKDNQSHLKADKNQSTKQKDTIKTTLLKKLMCSMQHPPSWLIHWFNLYTKLNSILTQYRSSEVKNHGFILIDNHTLSGFQFILNQYGCIVYHKELKRITVTTYNQFLTWKDISLSRLNVCLITWISNCLNTLNKSLGLRCGFNNVILTQLFLYGDCILKLFHNEGFYIIKEIEGFIMSLILNITEEDQFRKRFYNSMLNNITDAANKAQKNLLSRVCHTLLDKTISDNIINGRWIILLSKFLKLIKLAGDNNLNNLSELYFLFRIFGHPMVDERQAMDAVKVNCNETKFYLLSSLSMLRGAFIYRIIKGFVNNYNRWPTLRNAIVLPLRWLTYYKLNTYPSLLELTERDLIVLSGLRFYREFRLPKKVDLEMIINDKAISPPKNLIWTSFPRNYMPSHIQNYIEHEKLKFSDSDKSRRVLEYYLRDNKFNECDLHNCVVNQSYLNNPNHVVSLTGKERELSVGRMFAMQPGMFRQVQILAEKMIAENILQFFPESLTRYGDLELQKILELKAGISNKSNRYNDNYNNYISKCSIITDLSKFNQAFRYETSCICSDVLDELHGVQSLFSWLHLTIPHVTIICTYRHAPPYIKDHIVDLNNVDEQSGLYRYHMGGIEGWCQKLWTIEAISLLDLISLKGKFSITALINGDNQSIDISKPVRLMEGQTHAQADYLLALNSLKLLYKEYAGIGHKLKGTETYISRDMQFMSKTIQHNGVYYPASIKKVLRVGPWINTILDDFKVSLESIGSLTQELEYRGESLLCSLIFRNVWLYNQIALQLKNHALCNNKLYLDILKVLKHLKTFFNLDNIDTALTLYMNLPMLFGGGDPNLLYRSFYRRTPDFLTEAIVHSVFILSYYTNHDLKDKLQDLSDDRLNKFLTCIITFDKNPNAEFVTLMRDPQALGSERQAKITSEINRLAVTEVLSTAPNKIFSKSAQHYTTTEIDLNDIMQNIEPTYPHGLRVVYESLPFYKAEKIVNLISGTKSITNILEKTSAIDLTDIDRATEMMRKNITLLIRILPLDCNRDKREILSMENLSITELSKYVRERSWSLSNIVGVTSPSIMYTMDIKYTTSTIASGIIIEKYNVNSLTRGERGPTKPWVGSSTQEKKTMPVYNRQVLTKKQRDQIDLLAKLDWVYASIDNKDEFMEELSIGTLGLTYEKAKKLFPQYLSVNYLHRLTVSSRPCEFPASIPAYRTTNYHFDTSPINRILTEKYGDEDIDIVFQNCISFGLSLMSVVEQFTNVCPNRIILIPKLNEIHLMKPPIFTGDVDIHKLKQVIQKQHMFLPDKISLTQYVELFLSNKTLKSGSNVNSNLILAHKISDYFHNTYILSTNLAGHWILIIQLMKDSKGIFEKDWGEGYITDHMFINLKVFFNAYKTYLLCFHKGYGRAKLECDMNTSDLLCVLELIDSSYWKSMSKVFLEQKVIKYILSQDASLHRVKGCHSFKLWFLKRLNVAEFTVCPWVVNIDYHPTHMKAILTYIDLVRMGLINIDRIYIKNKHKFNDEFYTSNLFYINYNFSDNTHLLTKHIRIANSELESNYNKLYHPTPETLENILTNPVKSNGKKTLSDYCIGKNVDSIMLPSLSNKKLIKSSTMIRTNCSRQDLYNLFPTVVIDKIIDHSGNTAKSNQLYTTTSHQISLVHNSTSLYCMLPWHHINRFNFVFSSTGCKISIEYILKDLKIKDPNCIAFIGEGAGNLLLRTVVELHPDIRYIYRSLKDCNDHSLPIEFLRLYNGHINIDYGENLTIPATDATNNIHWSYLHIKFAEPISLFVCDAELPVTVNWSKIIIEWSKHVRKCKYCSSVNKCTLIVKYHAQDDIDFKLDNITILKTYVCLGSKLKGSEVYLVLTIGPANVFPVFNVAQNAKLILSRTKNFIMPKKADKESIDANIKSLIPFLCYPITKKGINTALSKLKSVVSGDILSYSIAGRNEVFSNKLINHKHMNILKWFNHVLNFRSTELNYNHLYMVESTYPHLSELLNSLTTNELKKLIKITGSLLYNFYNE

(19) United States
(12) Patent Application Publication
(10) Pub. No.: US 2026/0096999 A9
(43) Pub. Date: Apr. 9, 2026

(54) RESPIRATORY SYNCYTIAL VIRUS (RSV) POLYANHYDRIDE NANOPARTICLE VACCINE

(71) Applicant: University of Iowa Research Foundation, Iowa City, IA (US)

(72) Inventors: Steven M. Varga, Coralville, IA (US); Kevin L. Legge, North Liberty, IA (US)

(21) Appl. No.: 17/674,683

(22) Filed: Feb. 17, 2022

Related U.S. Application Data
(63) Continuation of application No. 16/666,214, filed on Oct. 28, 2019, now abandoned.
(60) Provisional application No. 62/752,006, filed on Oct. 29, 2018.

(51) Int. Cl. C12N 15/113; A61K 9/00; A61K 31/7105; A61K 31/713

(52) U.S. Cl. CPC A61K 9/5146; A61K 9/0048; A61K 31/7105; A61K 31/713; A61K 39/155; A61K 39/39; A61P 31/14; C12N 7/00; C12N 15/113; C12N 15/1136; A61K 2039/55561; C12N 2310/14; C12N 2310/321; C12N 2320/30; C12N 2320/31; C12N 2760/18534

(57) ABSTRACT

Disclosed are compositions and methods for vaccinating susceptible individuals against infection by respiratory syncytial virus (RSV). The disclosed compositions include vaccine compositions comprising an effective amount of respiratory syncytial virus (RSV) F protein in a pre-fusion stabilized form and/or M protein incorporated into biodegradable polyanhydride polymer particles for inducing an immune response against RSV. The vaccine compositions also may include a suitable adjuvant.

### CROSS-REFERENCE TO RELATED PATENT APPLICATIONS

The present application claims the benefit of priority under 35 U.S.C. § 119(e) to U.S. Provisional Application No. 62/752,006, filed on Oct. 29, 2018, the content of which is incorporated herein by reference in its entirety.

### STATEMENT REGARDING FEDERALLY SPONSORED RESEARCH OR DEVELOPMENT

This invention was made with government support under AI124093 awarded by the National Institutes of Health. The government has certain rights in the invention.

### BACKGROUND

The field of the invention relates to compositions and methods for inducing an immune response against respiratory syncytial virus (RSV). In particular, the field of the invention relates to compositions and methods for vaccinating susceptible individuals against infection by RSV by administering particulate vaccines comprising RSV structural components or variants thereof and optionally an adjuvant.

Respiratory syncytial virus (RSV) is the leading cause of lower respiratory infections in young children, resulting in 34 million new RSV infections each year, and approximately 125,000 hospitalizations annually in the United States alone. RSV reinfection is common in children, and even adults can be susceptible to repeated infection due to short-lived and incomplete protective immunity after natural infection. Especially vulnerable populations include premature babies, the elderly, people with heart or lung disease, and people with a very weak immune system. There are no effective vaccines for RSV, and work in the field has been slow since two children died during an RSV vaccine test in 1966.

RSV is an RNA virus with a genome that expresses eleven (11) known proteins. One of these, F protein, causes the virion membrane to fuse with the target cell's membrane. F proteins are targeted by antibodies after infection and are the major target for antiviral drug development. Fusion causes a confirmation change that obscures the major F antigenic site, site Ø. As such, RSV vaccines that induce immune response against Ø and other antigens of RSV such as M protein are desirable.

### SUMMARY

Disclosed are compositions and methods for vaccinating susceptible individuals against infection by respiratory syncytial virus (RSV). The disclosed compositions include vaccine compositions comprising an effective amount of respiratory syncytial virus (RSV) F protein in a pre-fusion stabilized form and/or M protein incorporated into biodegradable polyanhydride polymer particles for inducing an immune response against RSV. The vaccine compositions also may include a suitable adjuvant.

### BRIEF DESCRIPTION OF THE FIGURES

FIG. 1. Prime/boost nanoparticle vaccination strategy with prefusion RSV F protects against RSV-induced disease. BALB/c mice were primed with 500 μg of the indicated nanoparticle formulation i.n. on day 0, and boosted with 500 μg i.n. on day 28. Control mice (labeled RSV) were administered PBS i.n. on both prime and boost days. On day 56, all mice were challenged with 4.8×10⁶ PFU RSV-A2 i.n. and assessed for (A) weight loss, (B) Penh, and (C) EF50. Data are represented as mean±SEM of 3 independent experiments (n=11 mice for prefusionF+M+CpG, n=12 mice for RSV, CpG, M+CpG, and postfusion F+CpG, n=10 mice for prefusion F+CpG). Asterisks represent significance between RSV and prefusion F+M+CpG, pound symbols represent significance between RSV and prefusion F+CpG, and ‡ represents significance between RSV and postfusion F+CpG as determined by a 2-way ANOVA with a Dunnett' s post hoc test. ^{*/#/‡}p<0.05, ^{**/##/‡‡}p<0.01, ^{***/###/‡‡‡}p<0.001

FIG. 2. Prime/boost nanoparticle vaccination with prefusion RSV F reduces RSV N gene viral copy numbers. BALB/c mice were primed with 500 μg of the indicated nanoparticle formulation i.n. on day 0, and boosted with 500 μg i.n. on day 28. Control mice (labeled RSV) were administered PBS i.n. on both prime and boost days. On day 56, all mice were challenged with 4.8×10⁶ PFU RSV-A2 i.n. 4 days post-challenge lungs were harvested for RNA. RT-PCR was performed to determine RSV N gene copy numbers. Data represent mean±SEM of 2 independent experiments (n=8 mice for postfusion F+CpG, M+CpG, and prefusion F+CpG, n=7 mice for RSV, n=6 mice for prefusion F+M+CpG, n=4 mice for CpG). Statistical significance was determined by a one-way ANOVA with a Tukey's post hoc test. ^{*}p<0.05, ^{**}p<0.01

FIG. 3. Initial priming vaccination with prefusion RSV F induces lung-resident CD4 and CD8 T cells. BALB/c mice were primed with 500 μg of the indicated nanoparticle formulation or PBS (naïve) i.n. on day 0. Lungs and spleen were harvested on day 8 and analyzed by flow cytometry. Frequency of (A) activated (CD11a^{hi}CD49⁺) CD4 T cells and (B) activated (CD11a^{hi}CD8^{lo}) CD8 T cells. Number of i.v.⁻ (C) CD4 and (D) CD8 T cells. Number of activated i.v⁻ (E) CD103⁻CD69⁺ CD4 T cells and (F) CD103⁺CD69⁺ CD8 T cells. (G) Representative flow cytometry plots of germinal center B cells (CD19⁺CDB220⁺Fas⁺GL-7⁺). Data are represented as mean±SEM from a single experiment (n=4 mice for naïve, preF+CpG, preF+M+CpG, n=3 mice for CpG, PreF, PreF+M). Statistical significance was determined by a 2-way ANOVA with Tukey's post hoc test (A and B). ^{*}p<0.05, ^{**}p<0.01, ^{***}p<0.001

FIG. 4. Prime/boost nanoparticle vaccination induces RSV-specific antibodies in serum. BALB/c mice were primed with 500 μg of the indicated nanoparticle formulation i.n. on day 0, and boosted with 500 μg i.n. on day 28. Control mice (labeled naïve) were administered PBS i.n. on both prime and boost days. On day 14 and 56 serum was assessed for (A,D) total RSV-specific IgG, (B,E) IgG1, or (C,F) IgG2a. Data are represented as mean±SEM from a single experiment (n=4 mice). Asterisks represent significance between naive and prefusion F+M+CpG, and ‡ represents significance between naive and postfusion F+CpG as determined by a 2-way ANOVA with Dunnett's post hoc test. ^{*/#/‡}p<0.05, ^{**/##/‡‡}p<0.01, ^{***/###/‡‡‡}p<0.001

FIG. 5. Prime/boost nanoparticle vaccination with prefusion RSV F induces lung-resident B cells, CD4, and CD8 T cells. BALB/c mice were primed with 500 μg of the indicated nanoparticle formulation or PBS (naïve) i.n. on day 0, and boosted with 500 μg i.n. on day 28. Lung and spleen were harvested on day 42 and analyzed by flow cytometry. (A) Number of i.v.⁻ B cells. Number of activated i.v.⁻ (B) CD103⁻CD69⁺ CD4 T cells and (C) CD103⁺CD69⁺CD8 T cells. Number of activated (D) IFN-γ⁺ CD4 T cells and (E) IL-5⁺CD4 T cells following stimulation with PMA/ionomycin. Data are represented as mean±SEM from a single experiment (n=4 mice). Asterisks represent significance between naive and prefusion F+M+CpG, and ‡ represents significance between naive and postfusion F+CpG as determined by a one-way ANOVA with Tukey's post hoc test. ^{*}p<0.05, ^{**}p<0.01, ^{***}p<0.001.

FIG. 6. Prime/boost nanoparticle vaccination with prefusion RSV F protects against RSV-induced weight loss and pulmonary dysfunction. (A-B) BALB/c mice were primed with 500 μg of the indicated nanoparticle formulation i.n. on day 0, and challenged with 4.8×10⁶ PFU RSV-A2 i.n. on day 28. No vaccine mice were administered PBS i.n. at the prime. Groups were monitored daily for (A) weight loss and (B) airway obstruction (Penh). (C-D) BALB/c mice were primed with 500 μg of the indicated nanoparticle formulation i.n. on day 0, and boosted with 500 μg i.n. on day 28. No vaccine mice were administered PBS i.n. on both prime and boost days. All mice were challenged with 4.8×10⁶ PFU RSV-A2 i.n. on (C-D) day 56 or (E-F) day 100 and assessed for weight loss and airway obstruction (Penh). Asterisks represent significance between no vaccine and preF+CpG and pound symbols represent significance between CpG and preF+CpG as determined by (A-B) Student t test, or (C-F) 2-way ANOVA with Dunnett's post hoc test. ^{*/#}p<0.05, ^{**/##}p<0.01, ^{***/###}p<0.001

FIG. 7. Prime/boost nanoparticle vaccination with prefusion RSV F reduces infectious RSV particles. (A-B) BALB/c mice were primed with 500 μg of the indicated nanoparticle formulation i.n. on day 0, and boosted with 500 μg i.n. on day 28. No vaccine mice were administered PBS i.n. on both prime and boost days. RSV immune mice received 4.8×10⁶ PFU RSV-A2 i.n. at the prime and PBS i.n. at the boost. On (A) day 56 or (B) day 100, all mice were challenged with 4.8×10⁶ PFU RSV-A2 i.n and infectious plaque-forming units (pfu) were quantified in the lung on (A) day 2 or (A and B) day 4 by plaque assay. (C) BALB/c mice were primed with 500 μg of the indicated nanoparticle formulation i.n. on day 0, and challenged with 4.8×10⁶ PFU RSV-A2 i.n. on day 28. No vaccine mice were administered PBS i.n., and RSV immune mice received 4.8×10⁶ PFU RSV-A2 i.n. at the prime. On day 28, all mice were challenged with 4.8×10⁶ PFU RSV-A2 i.n and infectious pfu were quantified in the lung on day 4 by plaque assay. Statistical significance was determined by (A) 2-way ANOVA or (B-C) one-way ANOVA with a Tukey's post hoc test. ^{*}p<0.05, ^{**}p<0.01, ^{***}p<0.001.

FIG. 8. Prime/boost nanoparticle vaccination with prefusion RSV F induces activated T cells in the lungs. BALB/c mice were primed with 500 μg of the indicated nanoparticle formulation i.n. on day 0, and boosted with 500 μg i.n. on day 28. No vaccine mice were administered PBS i.n. on both prime and boost days. RSV immune mice received 4.8×10⁶ PFU RSV-A2 i.n. at the prime and PBS i.n. at the boost. Lungs and spleens were harvested on day 42 and analyzed by flow cytometry. Frequency of (A) activated (CD11a^{hi}CD49d⁺) CD4 T cells and (B) activated)(CD11a^{hi}CD8^{lo}) CD8 T cells. Statistical significance was determined by 2-way ANOVA with Tukey's post hoc test. ^{**}p<0.01, ^{***}p<0.001.

FIG. 9. Prime/boost nanoparticle vaccination with prefusion RSV F induces tissue-resident CD4 and CD8 T cells. BALB/c mice were primed with 500 μg of the indicated nanoparticle formulation i.n. on day 0, and boosted with 500 μg i.n. on day 28. No vaccine mice were administered PBS i.n. on both prime and boost days. RSV immune mice received 4.8×10⁶ PFU RSV-A2 i.n. at the prime and PBS i.n. at the boost. Lungs were harvested on day 42 and analyzed by flow cytometry. Number of activated CD45 intravascular antibody (i.v.⁻) (A) CD103⁻CD69⁺ CD4 T cells and (B) CD103⁺CD69⁺ CD8 T cells. Number of activated IFN-γ⁺ (C) CD4 and (D) CD8 T cells following stimulation with PMA/ionomycin. Number of (E) activated F₈₅₋₉₃ tetramer⁺ CD8 T cells and (F) i.v.⁻ activated F₈₅₋₉₃ tetramer⁺CD8 T cells. Statistical significance was determined by one-way ANOVA (C-F) with Tukey's post hoc test. ^{*}p<0.05, ^{**}p<0.01, ^{***}p<0. 001.

FIG. 10. BALB/c mice were primed with 500 μg of the indicated nanoparticle formulation i.n. on day 0, and boosted with 500 μg i.n. on day 28. No vaccine mice were administered PBS i.n. on both prime and boost days. RSV immune mice received 4.8×10⁶ PFU RSV-A2 i.n. at the prime and PBS i.n. at the boost. (A-B) On (A) day 56 or (B) day 100, all mice were challenged with 4.8×10⁶ PFU RSV-A2 i.n and monitored daily for the respiratory parameter EF50. (C) On day 56, all groups were challenged with 4.8×10⁶ PFU RSV-A2 i.n and RSV N gene copy numbers per lung on day 4 were determined by RT-PCR. Asterisks represent significance between no vaccine and preF+CpG and pound symbols represent significance between CpG and preF+CpG as determined by (A-B) 2-way ANOVA or (C) one-way ANOVA with Tukey's post hoc test. ^{*/#}p<0.05, ^{**/##}p<0.01, ^{***/###}p<0.001

FIG. 11. Illustration of Pre-F protein triggering, fold-back, and facilitating membrane fusion between a target cell and a virion.

FIG. 12. Illustrative vaccination schedule.

### DETAILED DESCRIPTION

The disclosed subject matter further may be described utilizing terms as defined below.

Unless otherwise specified or indicated by context, the terms “a”, “an”, and “the” mean “one or more.” For example, “a protein” and “an adjuvant” should be interpreted to mean “one or more proteins” and “adjuvants,” respectively.

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus ≤10% of the particular term and “substantially” and “significantly” will mean plus or minus >10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion additional components other than the components recited in the claims. The term “consisting essentially of” should be interpreted to be partially closed and allowing the inclusion only of additional components that do not fundamentally alter the nature of the claimed subject matter.

The terms “subject,” “patient,” and “individual” may be used interchangeably herein. A subject may be a human subject. A subject may refer to a human subject having or at risk for infection by respiratory syncytial virus (RSV). Human RSV (HRSV) is the leading cause of severe respiratory infections in neonates and children. HRSV belongs to the Orthopneumovirus genus within the Pneumoviridae family of viruses. HRSV is enveloped and has a negative-sense, single-stranded RNA genome of approximately 15 kb that encodes 11 viral proteins which include the F (fusion) protein that is a transmembrane protein of the virus and the M (matrix) protein that is a core protein of the virus. The HRSV-GNA435/11 strain has been sequenced and includes 15,277 bp encoding 11 viral proteins. (See Lee et al., Complete Genome Sequence of Human Respiratory Syncytial Virus Genotype A with a 72-Nucleotide Duplication in the Attachment Protein G Gene, J. Virol., December 2012, Vol. 86, No. 24, p. 13810-13811, the content of which is incorporated herein by reference in its entirety). The corresponding DNA sequence of the HRSV genome is provided herein as SEQ ID NO:1. The amino acid sequences of the NS1, NS2, N, M, P, G, F, SH, MS-1, MS-2, and L genes are provided herein as SEQ ID NOs: 2-12, respectively.

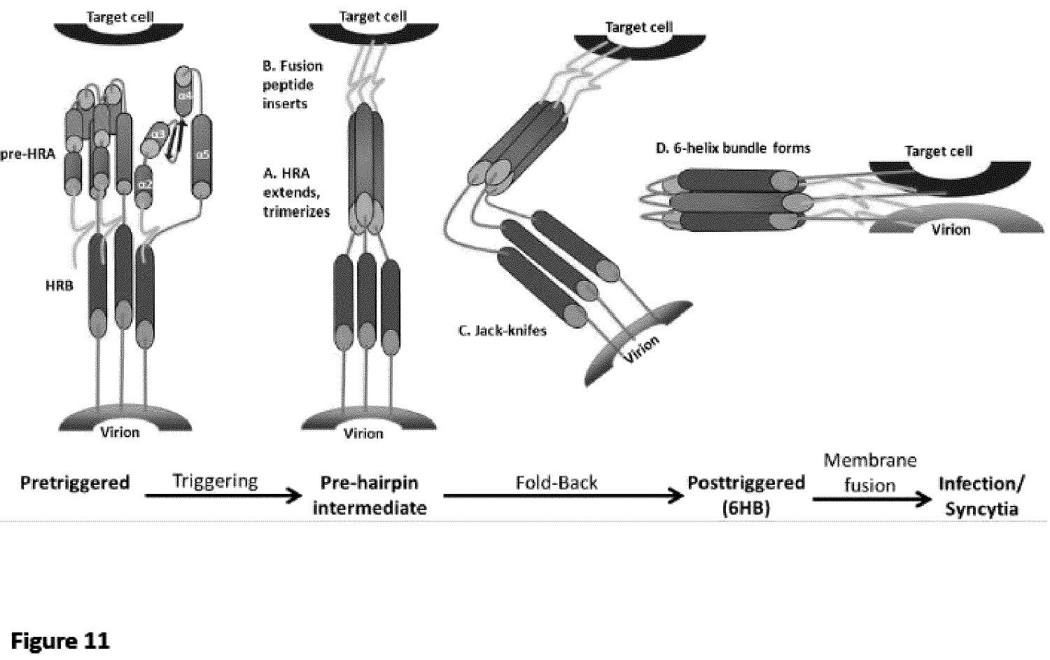

The disclosed compositions may include an effective amount of respiratory syncytial virus (RSV) F protein in a pre-fusion stabilized form incorporated into biodegradable polyanhydride polymer particles. The RSV F protein is a class I viral fusion glycoprotein that mediates membrane fusion between RSV and a host cell during viral entry. The F protein undergoes a conformation change from a “pre-fusion” to a “post-fusion” state during virus entry. (See FIG. 11). Some neutralizing antibodies against RSV have been shown to bind to a site called (Ø), which is present only in the pre-fusion form of F protein. Recombinant stabilized pre-fusion forms of F protein have been disclosed in the art and have been shown to be more effective at inducing neutralizing antibodies. (See Steff et al., Nat. Commun., 8:1085 (2017), pages 1-10; Blais et al., J. Virol. 91, e02437-16 (2017); McLellan et al., Science 342, 593-598 (2013); Krarup et al., Nat. Commun. 6, 8143 (2015); and Joyce et al., Nat. Struct. Mol. Biol. 23, 611-820 (2016); the contents of which are incorporated herein by reference in their entireties. Pre-fusion stabilized forms of F protein that are disclosed in the art include forms called DS-Cav1, DS-TriC, Cav-1-TriC, and DS-Cav1-TriC (see McLellan et al., Science 342, 593-598 (2013)), Pre-F-GCN4t (see Blais et al., J. Virol. 91, e02437-16 (2017)), and SC-DM, and SC-TM Krarup et al., Nat. Commun. 6, 8143 (2015)).

The disclosed compositions may include an effective amount of respiratory syncytial virus (RSV) M protein incorporated into biodegradable polyanhydride polymer particles. The M protein of RSV is a core protein that may be important for inducing a cell-mediated response against RSV infection.

Optionally, the disclosed compositions may include an effective amount of other respiratory syncytial virus (RSV) proteins incorporated into biodegradable polyanhydride polymer particles. Other proteins or RSV may include one or more of the RSV proteins selected from NS1, NS2, N, P, SH, G, M2-1, M2-2, L, or any combinations thereof.

As used herein, the phrase “effective amount” shall mean that dosage that provides the specific immunological response for which a composition comprising that effective amount is administered in a significant number of subjects. For example, an effective amount of an antigen may include that amount which when administered to a vaccinee induces an immune response in the vaccinee, preferably a protective immune response against the pathogen from which the antigen is derived. An effective amount of an antigen that is administered to a particular patient in a particular instance will not always be effective in treating the conditions/diseases described herein, even though such dosage is deemed to be a therapeutically effective amount by those of skill in the art.

The compositions disclosed herein may be formulated as vaccine compositions for administration to a subject in need thereof. Such compositions can be formulated and/or administered in dosages and by techniques well known to those skilled in the medical arts taking into consideration such factors as the age, sex, weight, and condition of the particular patient, and the route of administration.

The compositions may include pharmaceutical solutions comprising carriers, diluents, excipients, and surfactants as known in the art. Further, the compositions may include preservatives. The compositions also may include buffering agents.

The disclosed compositions typically include biodegradable particles. The biodegradable particles typically have an average effective diameter of less than about 5, 4, 3, 2, 1, 0.5, 0.1, 0.05, or 0.01 μM, or the biodegradable particles have an average effective diameter within a range bounded by any of these values (e.g., 0.1-2 μM).

In some embodiments, the disclosed particles may be phagocytosed by antigen presenting cells, such as macrophage and dendritic cells, when the disclosed particles are administered as an immunogenic composition or vaccine formulation to a subject in need thereof. Preferably, the disclosed particles have an effective average diameter to permit phagocytosis by antigen presenting cells. Particles larger than about 5 microns are unlikely to be phagocytosed by antigen presenting cells and preferably the particles have an effective average diameter of less than about 4 microns or more preferably the particles have an effective average diameter of less than about 3 microns.

The disclosed particles typically are biodegradable as would be understood in the art. The term “biodegradable” describes a material that is capable of being degraded in a physiological environment into smaller basic components by biochemical reactions and/or physical reactions. The term “biodegradable” may be used herein interchangeably with the term “bioerodible.” Preferably, the biodegradable particles are degraded (or eroded) into smaller basic components are innocuous. For example, a biodegradable polymer may be degraded into basic components that include, but are not limited to, water, carbon dioxide, sugars, organic acids (e.g., tricarboxylic or amino acids), and alcohols (e.g., glycerol or polyethylene glycol).

The disclosed compositions typically include biodegradable polyanhydride polymer particles which may include particles comprising or formed from homopolymers or copolymers. Biodegradable polyanhydride homopolymer and copolymer particles are known in the art. (See, e.g., U.S. Pat. Nos. 8,173,104 and 7,858,093, the contents of which are incorporated herein by reference in their entireties). In some embodiments, the biodegradable polyanhydride polymer particles comprise a polymer formed from a 1,ω-bis(p carboxyphenoxy)(C₂-C₁₂)alkane, a 1,ω-bis(p-carboxyphenoxy)(C₂-C₁₂)dioxa-alkane, and a (C₅-C₂₀)alkanoic diacid. In particular, the biodegradable polyanhydride polymer particles may comprise a polymer formed from 1,6-bis(p-carboxyphenoxy)hexane (CPH), 1,8-bis(p -carboxyphenoxy)-3,6-dioxaoctance (CPTEG), and sebacic acid (SA). Where the biodegradable polyanhydride polymer particles are comprised of a polymer formed from CPH and CPTEG, the ratio of CPH and CPTEG may be modulated to prepare particles having a suitable release profile. In some embodiments, the biodegradable polyanhydride polymer particles comprise a polymer formed from CPH and CPTEG at a ratio CPH:CPTEG selected from 10:90, 20:80, 30:70, 40:60, 50:50, 60:40, 70:30, 80:20, and 90:10, or within a range bounded by any of these ratios (e.g., 80-60:20-40 CPH:CPTEG). In some embodiments, the biodegradable polyanhydride polymer particles may be formulated to enhance uptake by and activation of dendritic cells. (See Carrillo-Conde et al., Acta Biomaterialia 8 (2012) 3618-3628; the content of which is incorporated herein by reference in its entirety)

Other biodegradable materials that may be utilized to prepare the particles contemplated herein may include materials disclosed in one or more of U.S. Pat. Nos. 7,470,283; 7,390,333; 7,128,755; 7,094,260; 6,830,747; 6,709,452; 6,699,272; 6,527,801; 5,980,551; 5,788,979; 5,766,710; 5,670,161; and 5,443,458; and U.S. Published Application Nos. 20090319041; 20090299465; 20090232863; 20090192588; 20090182415; 20090182404; 20090171455; 20090149568; 20090117039; 20090110713; 20090105352; 20090082853; 20090081270; 20090004243; 20080249633; 20080243240; 20080233169; 20080233168; 20080220048; 20080154351; 20080152690; 20080119927; 20080103583; 20080091262; 20080071357; 20080069858; 20080051880; 20080008735; 20070298066; 20070288088; 20070287987; 20070281117; 20070275033; 20070264307; 20070237803; 20070224247; 20070224244; 20070224234; 20070219626; 20070203564; 20070196423; 20070141100; 20070129793; 20070129790; 20070123973; 20070106371; 20070050018; 20070043434; 20070043433; 20070014831; 20070005130; 20060287710; 20060286138; 20060264531; 20060198868; 20060193892; 20060147491; 20060051394; 20060018948; 20060009839; 20060002979; 20050283224; 20050278015; 20050267565; 20050232971; 20050177246; 20050169968; 20050019404; 20050010280; 20040260386; 20040230316; 20030153972; 20030153971; 20030144730; 20030118692; 20030109647; 20030105518; 20030105245; 20030097173; 20030045924; 20030027940; 20020183830; 20020143388; 20020082610; and 0020019661; the contents of which are incorporated herein by reference in their entireties.

Typically, the biodegradable particles disclosed herein are degraded in vivo at a degradation rate such that the particles lose no more than about 20%, 30%, 40%, 50%, 60%, 70%, 80%, 90%, 95%, or 99% of their initial mass (or within a range bounded by any of these values) after about 1, 2, 3, 4, 5, 6, 7, or 8 weeks or more post-administration (or within a range bounded by any of these values). The particles may comprise or may be formed from polymeric or non-polymeric biodegradable material. If the particles comprise polymeric material, typically the particles are degraded into biodegradable monomers. If the particles comprise non-polymeric material, typically the particles are degraded into biodegradable components.

The disclosed biodegradable particles may be prepared by methods known in the art including, but not limited to, spray-drying, precipitation, and grinding. In some embodiments, the biodegradable particles may be formed from a solution or suspension of a biodegradable material optionally in the presence of one or more additional agents such as adjuvants, apoptosis inhibitors, and/or antigens (e.g., by spray-drying the solution or suspension). As such, the biodegradable particles may comprise biodegradable material and optionally may comprise one or more additional agents such as adjuvants, apoptosis inhibitors, and/or antigens.

The disclosed biodegradable particles may be administered by various routes in order to induce a response in a subject. Routes of administration may include, but are not limited to, intranasal, pulmonary, oral, subcutaneous, intramuscular, and intravenous.

In some embodiments, the disclosed methods comprise administering a composition comprising biodegradable particles to induce an immune response in the subject. In other embodiments, the disclosed methods consist of administering a composition consisting of biodegradable particles to induce an immune response in the subject. The induced immune response may include an antibody response, a Th1 cell response and a CD8 CTL response.

The compositions disclosed herein optionally include an adjuvant. The term “adjuvant” refers to a compound or mixture that enhances an immune response. An adjuvant can serve as a tissue depot that slowly releases the antigen and also as a lymphoid system activator that non-specifically enhances the immune response. Examples of adjuvants which may be utilized in the disclosed compositions include but are not limited to, co-polymer adjuvants (e.g., Pluronic L121® brand poloxamer 401, CRL1005, or a low molecular weight co-polymer adjuvant such as Polygen® adjuvant), poly (I:C), R-848 (a Th1-like adjuvant), resiquimod, imiquimod, PAM3CYS, aluminum phosphates (e.g., AlPO₄), loxoribine, potentially useful human adjuvants such as BCG (Bacille Calmette-Guerin) and Corynebacterium parvum, CpG oligodeoxynucleotides (ODN), cholera toxin derived antigens (e.g., CTA1-DD), lipopolysaccharide adjuvants, complete Freund's adjuvant, incomplete Freund's adjuvant, saponin (e.g., Quil-A), mineral gels such as aluminum hydroxide, surface active substances such as lysolecithin, pluronic polyols, polyanions, peptides, oil or hydrocarbon emulsions in water (e.g., MF59 available from Novartis Vaccines or Montanide ISA 720), keyhole limpet hemocyanins, and dinitrophenol.

The compositions disclosed herein may include pharmaceutical compositions that are administered as vaccines. Typically, the pharmaceutical composition comprises an effective amount or concentration of an antigen for inducing a protective or therapeutic immune response against a disease, which may include, but is not limited to infection by a pathogen such as RSV. Inducing a protective or therapeutic immune response may include inducing an antibody response, as well as a CD4 and/or CD8 T cell immune response to one or more epitopes of a protein associated with a pathogen (e.g., a protein associated with RSV). Inducing a protective or therapeutic immune response may include inducing an antibody response, as well as a Th1 response and/or a CD8 T cell response to one or more epitopes of a protein associated with the pathogen. As utilized herein, a Th1-response may be characterized by cytokine production such as interferons (e.g., IFN-γ), tumor necrosis factor (e.g., TNF), and interleukins (e.g., IL-2). A Th1-response also may be characterized by increased killing efficiency of macrophages with respect to a pathogen and the proliferation of cytotoxic CD8⁺ cells against the pathogen. A Th1 response also may be characterized by the presence of opsonizing antibodies against the antigen.

Inducing a protective response may include inducing immunity against the pathogen, and in some embodiments, inducing protective immunity and/or sterilizing immunity against the pathogen. Inducing a therapeutic response may include reducing the pathogenic load of a subject, for example, as determined by measuring the amount of circulating pathogen before and after administering the composition. Inducing a therapeutic response may include reducing the degree or severity of at least one symptom of infection by the pathogen.

The presently disclosed methods may be utilized for inducing a protective or therapeutic immune response against disease by administering the pharmaceutical compositions disclosed herein (e.g., as immunogenic compositions or vaccines) to a subject in need thereof, which may include a human or non-human having or at risk for acquiring the disease. The methods may include administering a first pharmaceutical composition and optionally may include administering a second pharmaceutical composition to augment or boost an immunogenic response induced by the first pharmaceutical composition. The first and second pharmaceutical compositions may be the same or different. The optionally administered second pharmaceutical composition may be administered prior to, concurrently with, or after administering the first pharmaceutical composition. In some embodiments, the first composition is administered and then the second composition is administered after waiting at least about 1, 2, 3, 4, 5, or 6 weeks. The first composition (and the second composition) may be administered one or more times.

The presently disclosed compositions, kits, and methods may be utilized to protect against or treat infection by a pathogen. As used herein, a “pathogen” includes, but is not limited to a living microorganism such as bacteria, viruses, and fungi that cause disease in a host. As used herein, a “pathogen” includes respiratory syncytial virus (RSV).

The presently disclosed composition may be administered to potentiate or enhance an immune response. As used herein, “potentiating” or “enhancing” an immune response means increasing the magnitude and/or the breadth of the immune response. For example, the number of cells that recognize a particular epitope may be increased (“magnitude”) and/or the numbers of epitopes that are recognized may be increased (“breadth”). Preferably, an enhanced antibody response as well as an enhancement in CD4 and/or CD8 T-cell responses may be obtained by administering the pharmaceutical composition disclosed herein.

### Illustrative Embodiments

The following Embodiments are illustrative and should not be interpreted to limit the scope of the claimed subject matter.

Embodiment 1. A vaccine composition comprising an effective amount of respiratory syncytial virus (RSV) F protein in a pre-fusion stabilized form and/or M protein incorporated into biodegradable polyanhydride polymer particles for inducing an immune response against RSV.

Embodiment 2. The vaccine composition of embodiment 1, comprising an effective amount of RSV F protein in the pre-fusion stabilized form incorporated into the biodegradable polyanhydride polymer particles for inducing an immune response against RSV.

Embodiment 3. The vaccine composition of embodiment 1 or 2, wherein the F protein in a pre-fusion stabilized form is selected from the group consisting of DS-Cav1, DS-TriC, Vav-1-TriC, DX-Cav1-TriC, Pre-F-GCN4t, SC-DM, and SC-TM.

Embodiment 4. The vaccine composition of any of the foregoing embodiments, wherein the F protein in a pre-fusion stabilized form is DS-Cav1.

Embodiment 5. The vaccine composition of embodiment 1, comprising an effective amount of RSV M protein incorporated into the biodegradable polyanhydride polymer particles for inducing an immune response against RSV.

Embodiment 6 The vaccine composition of any of the foregoing embodiments, further comprising an adjuvant, optionally wherein the adjuvant is incorporated into the biodegradable polyanhydride polymer particles.

Embodiment 7. The vaccine composition of any of the foregoing embodiments, further comprising a CpG oligonucleotide, optionally wherein the CpG oligonucleotide is incorporated into the biodegradable polyanhydride polymer particles.

Embodiment 8. The vaccine composition of any of the foregoing embodiments, further comprising a CpG oligodeoxynucleotide (ODN), optionally wherein the CpG ODN is incorporated into the biodegradable polyanhydride polymer particles.

Embodiment 9. The vaccine composition of any of the foregoing embodiments, wherein the particles have an average effective diameter of less than about 5, 4, 3, 2, 1, 0.5, 0.1, 0.05, or 0.01 μM, or have an average effective diameter within a range bounded by any of these values (e.g., 2-0.1 μM).

Embodiment 10. The vaccine composition of any of the foregoing embodiments, wherein the vaccine composition induces an antibody response as well as a CD4 T cell response (e.g., a Th1-type response), a CD8 T cell response, or a combination thereof.

Embodiment 11. The vaccine composition of any of the foregoing embodiments, wherein biodegradable polyanhydride polymer particles comprise a polymer formed from a 1,ω-bis(p-carboxyphenoxy)(C₂-C₁₂)alkane, a 1,ω-bis(p-carboxyphenoxy)(C₂-C₁₂)dioxa-alkane, and a (C₅-C₂₀)alkanoic diacid.

Embodiment 12. The vaccine composition of any of the foregoing embodiments, wherein the biodegradable polyanhydride polymer particles comprise a polymer formed from 1,6-bis(p-carboxyphenoxy)hexane (CPH), 1,8-bis(p-carboxyphenoxy)-3,6-dioxaoctance (CPTEG), and sebacic acid (SA).

Embodiment 13. The vaccine composition of any of the foregoing embodiments, wherein the biodegradable polyanhydride polymer particles comprise a polymer formed from 1,6-bis(p-carboxyphenoxy)hexane (CPH), 1,8-bis(p-carb oxyphenoxy)-3,6-dioxaoctance (CPTEG), and sebacic acid (SA), wherein the polymer is formed from CPH and CPTEG at a ratio CPH:CPTEG selected from 10:90, 20:80, 30:70, 40:60, 50:50, 60:40, 70:30, 80:20, and 90:10, or within a range bounded by any of these ratios (e.g., 80-60:20-40 CPH: CPTEG).

Embodiment 14. The vaccine composition of any of the foregoing embodiments, wherein the biodegradable polyanhydride polymer particles comprise a polymer formed from 1,6-bis(p-carboxyphenoxy)hexane (CPH), 1,8-bis(p-carboxyphenoxy)-3,6-dioxaoctance (CPTEG), and sebacic acid (SA), wherein the polymer is formed from CPH and CPTEG at a ratio CPH:CPTEG of about 80:20.

Embodiment 15. The vaccine composition of any of the foregoing embodiments, further comprising an RSV protein selected from NS1, NS2, N, P, SH, G, M2-1, M2-2, L, or any combinations thereof.

Embodiment 16. A method comprising administering any of the foregoing vaccine compositions to a subject who is at risk for infection by RSV.

Embodiment 17. The method of embodiment 16, wherein after the vaccine composition is administered to the subject, the subject is protected against infection by RSV.

Embodiment 18. The method of embodiment 16 or 17, wherein the vaccine composition is administered by a route selected from intranasal, pulmonary, oral, subcutaneous, intramuscular, or intravenous.

### EXAMPLES

The following Examples are illustrative and should not be interpreted to limit the scope of the claimed subject matter.

### Title—Evaluation of a Polyanhydride-Based Nanoparticle Vaccine Utilizing RSV M and/or Profusion F

Reference is made to the poster presentation entitled “Evaluation of a polyanhydride-based nanoparticle vaccine utilizing RSV M and/or prefusion F,” presented at the 11^{th} International Respiratory Syncytial Virus Symposium, Oct. 31-Nov. 4, 2018, which is incorporated herein by reference in its entirety.

### Abstract

Respiratory syncytial virus (RSV) is the leading cause of lower respiratory tract infections in young children, resulting in 34 million new RSV infections each year, and approximately 125,000 hospitalizations annually in the United States alone. RSV reinfection is common in children, and even adults can be susceptible to repeated infection due to short-lived and incomplete protective immunity following natural infection. Despite the critical need, there is currently no licensed vaccine for RSV. Here we developed a polyanhydride nanoparticle-based vaccine utilizing the RSV matrix (M) protein and/or a prefusion-stabilized variant of the RSV fusion (F) protein (DS-Cav1). Our nanoparticle formulation offers several advantages over current vaccine strategies as it allows for continual, controlled release of the antigen and has been shown to induce robust T and B cell responses when paired with antigens from other pathogens. Inclusion of the prefusion conformation of RSV F within the vaccine allows for better exposure of the major RSV F antigenic site, termed site Ø, while the RSV M protein contains epitopes that are known targets of both CD4 and CD8 T cell responses. The RSV nanovaccine is composed of 20:80 1,8-bis(p-carboxyphenoxy)-3,6-dioxoctane (CPTEG):1,6-bis(p-carboxyphenoxy)hexane (CPH) copolymer nanoparticles encapsulating the M and DS-Cav1 prefusion F proteins with CpG 1668 ODN. Additional formulations include RSV F in its postfusion conformation with CpG, and DS-Cav1 protein and CpG. Controls consist of nanoparticles containing either pre-F or postfusion RSV F without CpG, and nanoparticles loaded with CpG alone. Here we evaluated the immunogenicity and the protective capacity of our nanoparticle vaccine strategy against a primary RSV infection.

### Methods

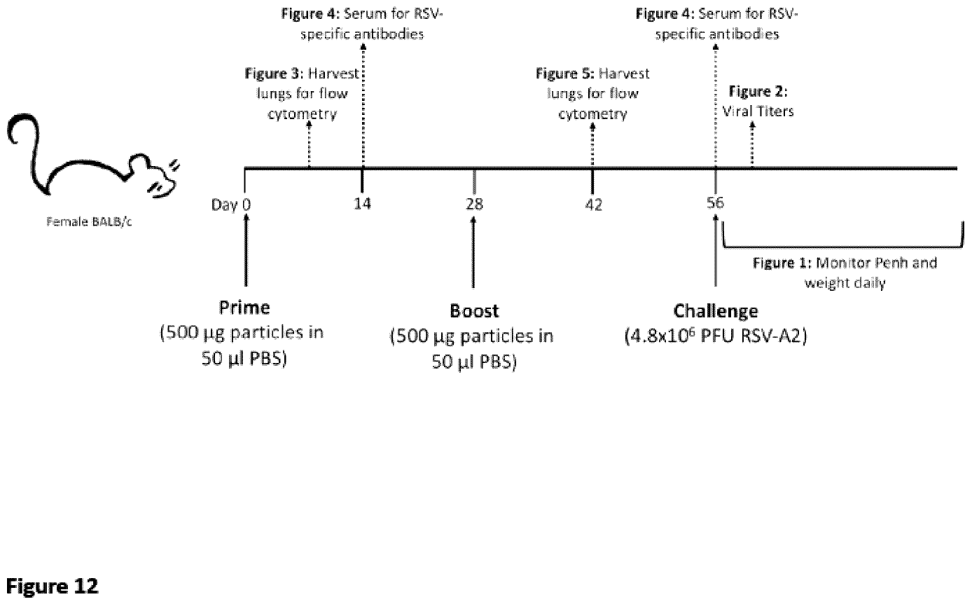

BALB/c mice were vaccinated intranasally with 500 μg of the nanoparticles in PBS administered at day 0 and subsequently boosted intranasally at day 28. (See FIG. 12). At day 56 the protective capacity of the nanoparticles was assessed by determining viral titers in the lungs of vaccinated mice following an RSV challenge. Using whole body plethysmography, we also measured airway function to determine the capacity of the RSV nanovaccine to protect against RSV-induced pulmonary injury. Lungs were harvested post-prime or post-boost to evaluate the T and B cell responses elicited by the nanoparticle vaccination.

### Results

The inventors are using the M+prefusion RSV F nanoparticle vaccine to determine the optimal vaccine regimen to provide protection against RSV-induced disease. Additionally, the inventors are investigating the ideal mixture of humoral and cell-mediated immunity necessary to establish long-term protective immunity against RSV. Information from these studies will be critical to establish optimal thresholds for protective immunity against RSV.

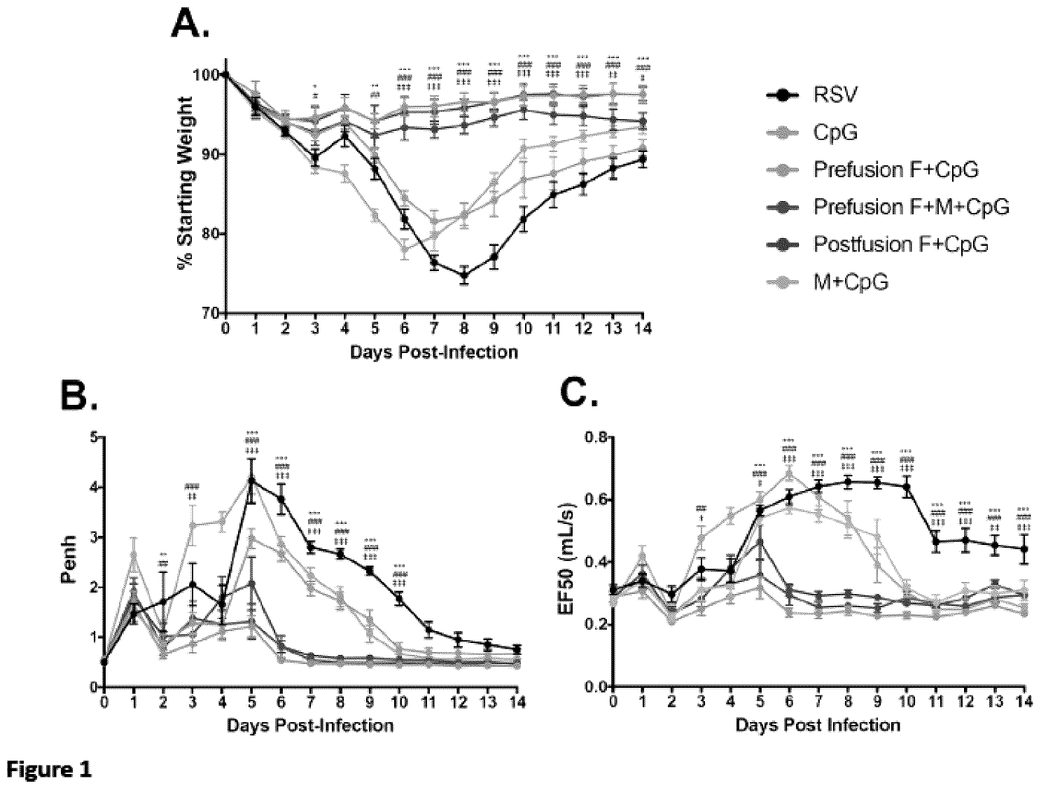

The results in FIG. 1 illustrate that a prime/boost nanoparticle vaccination strategy with prefusion RSV F protects against RSV-induced disease. BALB/c mice were primed with 500 μg of the indicated nanoparticle formulation i.n. on day 0, and boosted with 500 μg i.n. on day 28. Control mice (labeled RSV) were administered PBS i.n. on both prime and boost days. On day 56, all mice were challenged with 4.8×10⁶ PFU RSV-A2 i.n. and assessed for weight loss, Penh, and EF50.

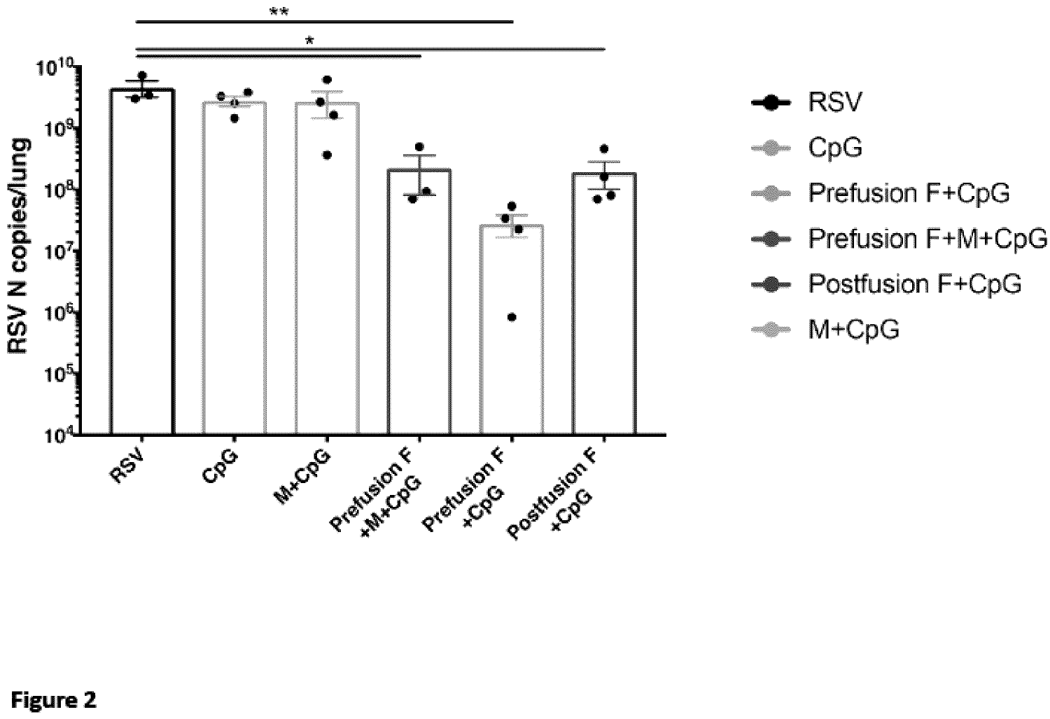

FIG. 2 illustrates that prime/boost nanoparticle vaccination with prefusion RSV F reduces RSV N gene viral copy numbers. BALB/c mice were primed with 500 μg of the indicated nanoparticle formulation i.n. on day 0, and boosted with 500 μg i.n. on day 28. Control mice (labeled RSV) were administered PBS i.n. on both prime and boost days. On day 56, all mice were challenged with 4.8×10⁶ PFU RSV-A2 i.n. 4 days post-challenge lungs were harvested for RNA. RT-PCR was performed to determine RSV N gene copy numbers.

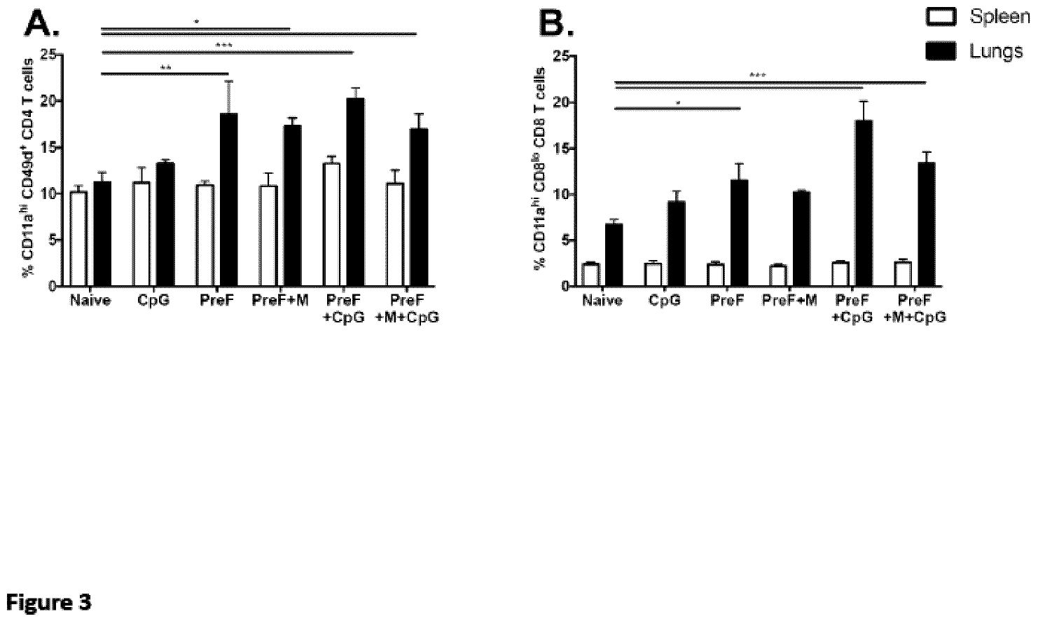

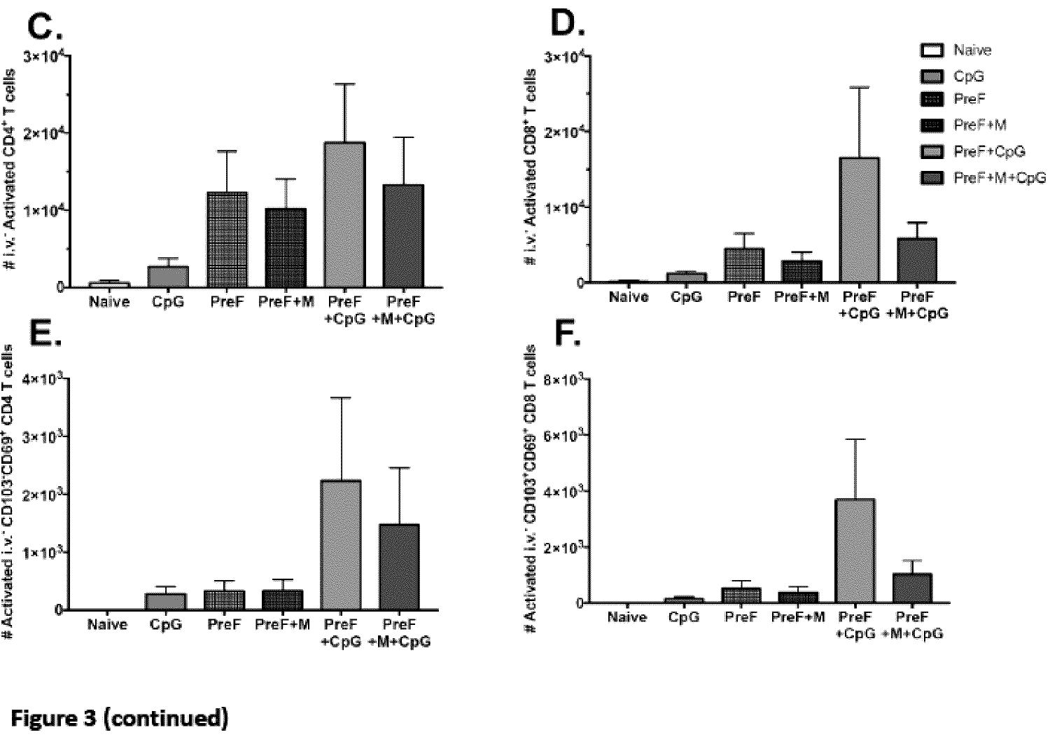

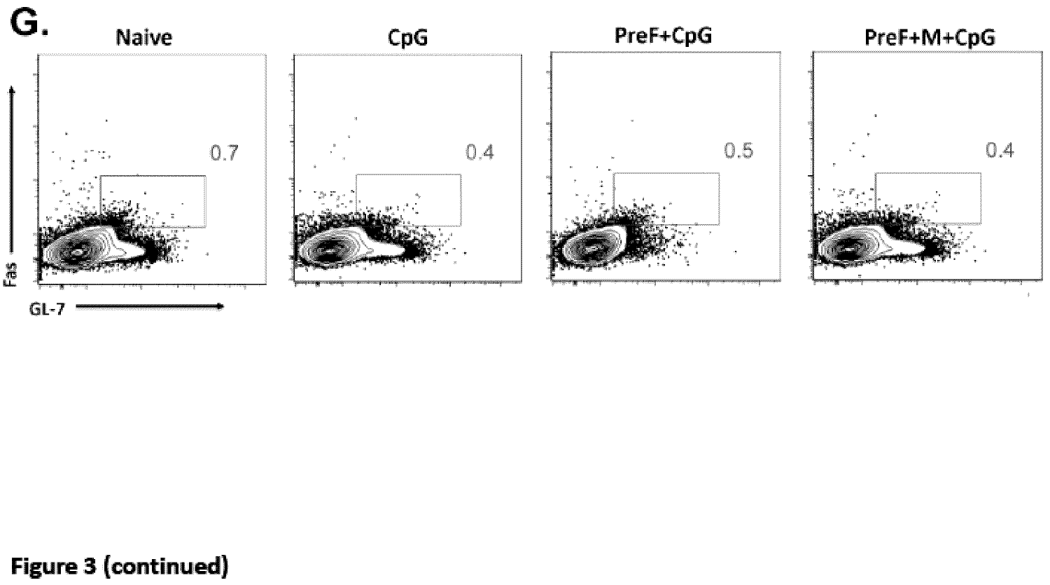

FIG. 3 illustrates that initial priming vaccination with prefusion RSV F induces lung-resident CD4 and CD8 T cells. BALB/c mice were primed with 500 μg of the indicated nanoparticle formulation or PBS (naïve) i.n. on day 0. Lungs and spleen were harvested on day 8 and analyzed by flow cytometry. The frequency of activated (CD11a^{hi}CD49d⁺) CD4 T cells and activated (CD11a^{hi}CD8^{lo}) CD8T cells was assessed as indicated in FIGS. 3A and 3B. The number of i.v.⁻CD4 and i.v.⁻CD8 T cells was assessed as indicated in FIGS. 3C and 3D. The number of activated i.v.⁻ CD103⁻CD69⁺ CD4 T cells and CD103⁺CD69⁺ CD8 T cells was assessed as indicated on FIGS. 3E and 3F. The representative flow cytometry plots of germinal center B cells (CD19⁺CDB220⁺Fas^{+GL-}7⁺) is illustrated in FIG. 3G.

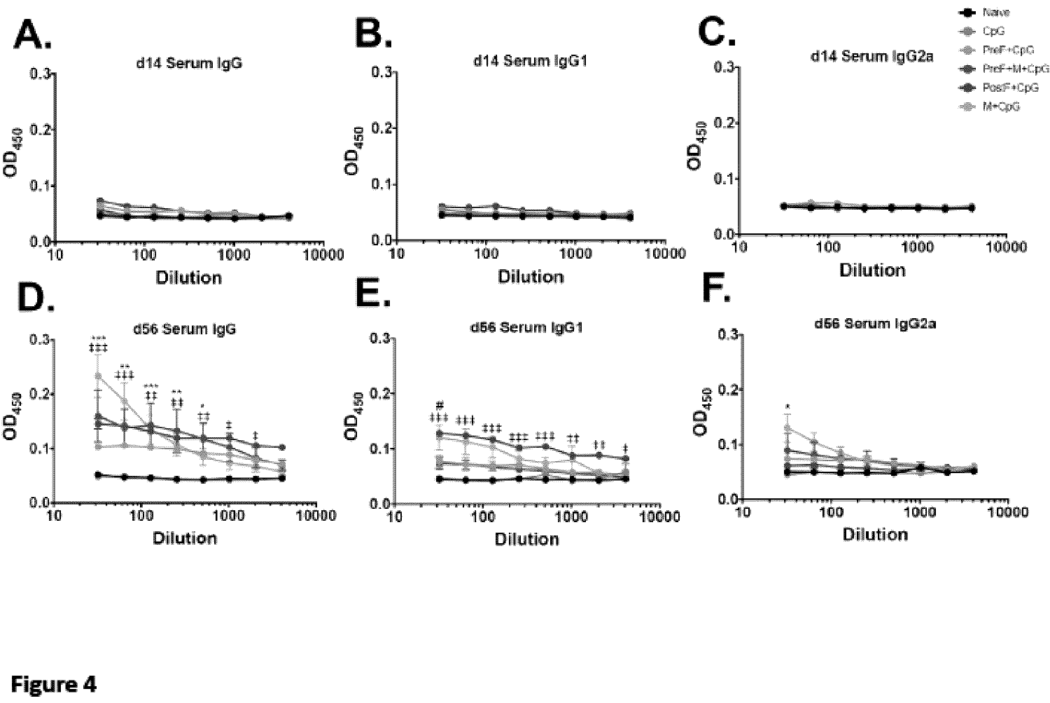

FIG. 4 illustrates that a prime/boost nanoparticle vaccination induces RSV-specific antibodies in serum. BALB/c mice were primed with 500 μg of the indicated nanoparticle formulation i.n. on day 0, and boosted with 500 μg i.n. on day 28. Control mice (labeled naïve) were administered PBS i.n. on both prime and boost days. On day 14 and 56 serum was assessed for total RSV-specific IgG, IgG1, or IgG2a.

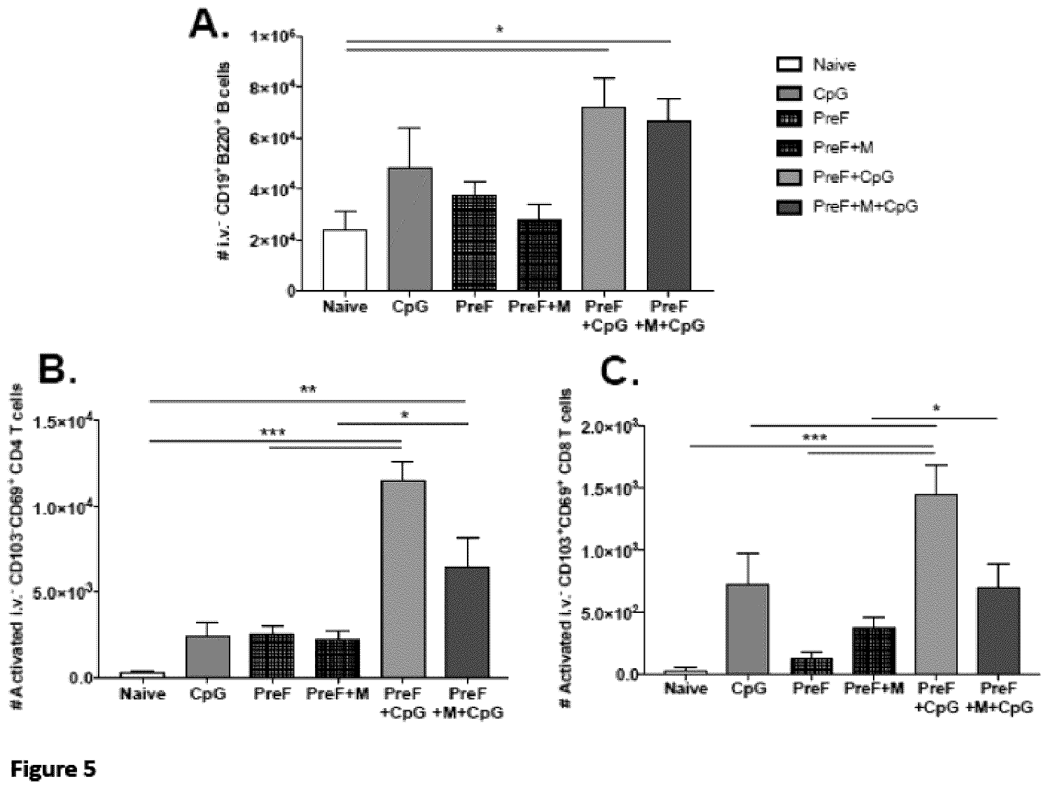

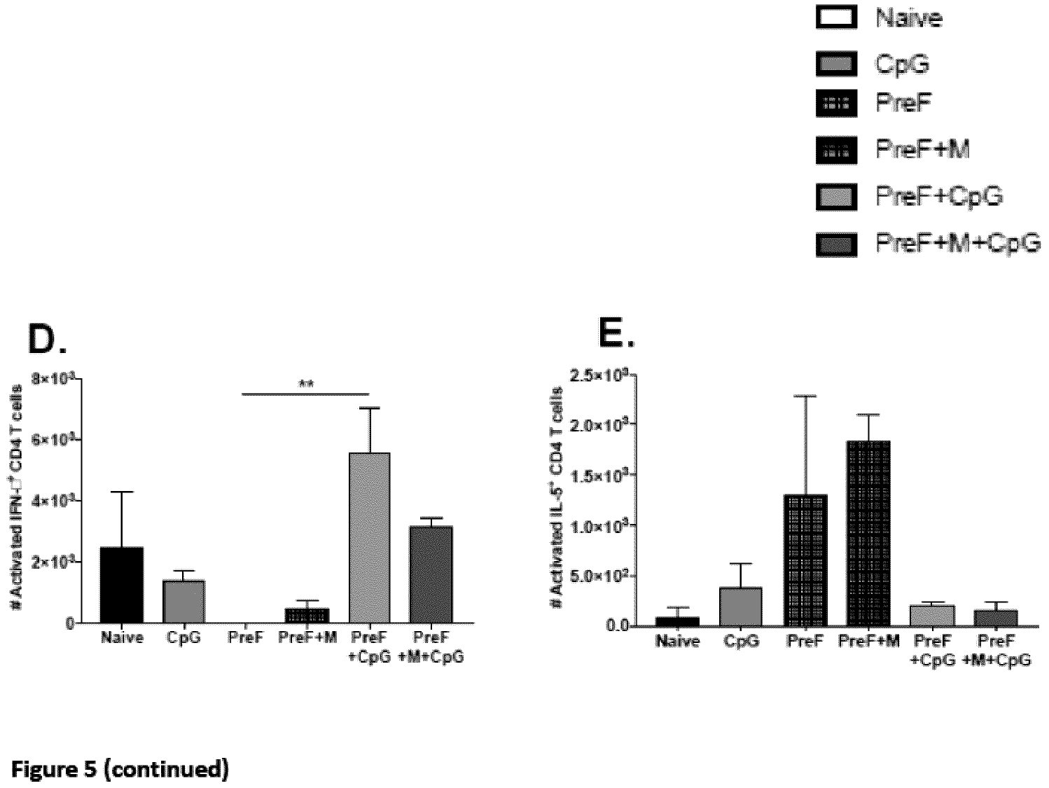

FIG. 5 illustrates that a prime/boost nanoparticle vaccination with prefusion RSV F induces lung-resident B cells, CD4, and CD8 T cells. BALB/c mice were primed with 500 μg of the indicated nanoparticle formulation or PBS (naïve) i.n. on day 0, and boosted with 500 μg i.n. on day 28. Lung and spleen were harvested on day 42 and analyzed by flow cytometry. We assessed the number of i.v.⁻ B cells, the number of i.v.⁻ activated CD103⁻CD69⁺ CD4 T cells and CD103⁺CD69⁺ CD8 T cells. We also assessed the number of activated IFN-γ⁺CD4 T cells and IL-5⁺ CD4 T cells following stimulation with PMA/ionomycin.

### Conclusion

Prime/boost nanoparticle vaccination with a prefusion RSV F nanoparticle-based vaccine reduced weight loss, pulmonary dysfunction, and viral copy numbers following an RSV challenge. Priming vaccination alone was shown to be sufficient to elicit i.v.⁻ tissue-resident CD4 and CD8 T cells. Prime/boost nanoparticle vaccination generated RSV-specific antibodies in serum, and prime/boost vaccination elicited IFN-γ⁺ CD4 T cells and tissue-resident T and B cells.

There are numerous benefits to the RSV vaccine as prepared by the inventors. First, the inventors' nanoparticle formulation allows for continual, controlled release of the antigen(s). Second, the inventors' nanoparticle formulation includes the F protein in a pre-fusion stabilized form, which allows for better exposure of the major RSV F antigenic site (Ø) to a vaccinee's immune system. Third, the inventors' nanoparticle formulation induces robust B and T cell responses due to inclusion of both of the F protein in a pre-fusion stabilized form and the M protein.

### Example 2—Protection Against RSV-Induced Disease by Nanoparticle Formulation Containing RSV Pre-F Protein and CpG Adjuvant

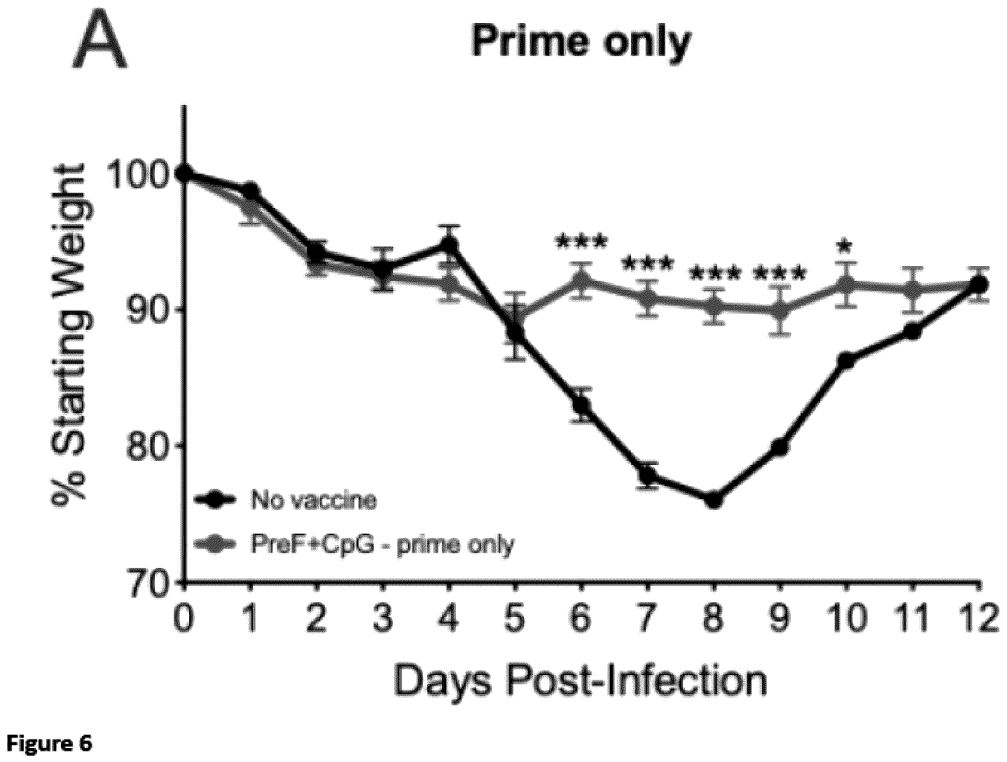

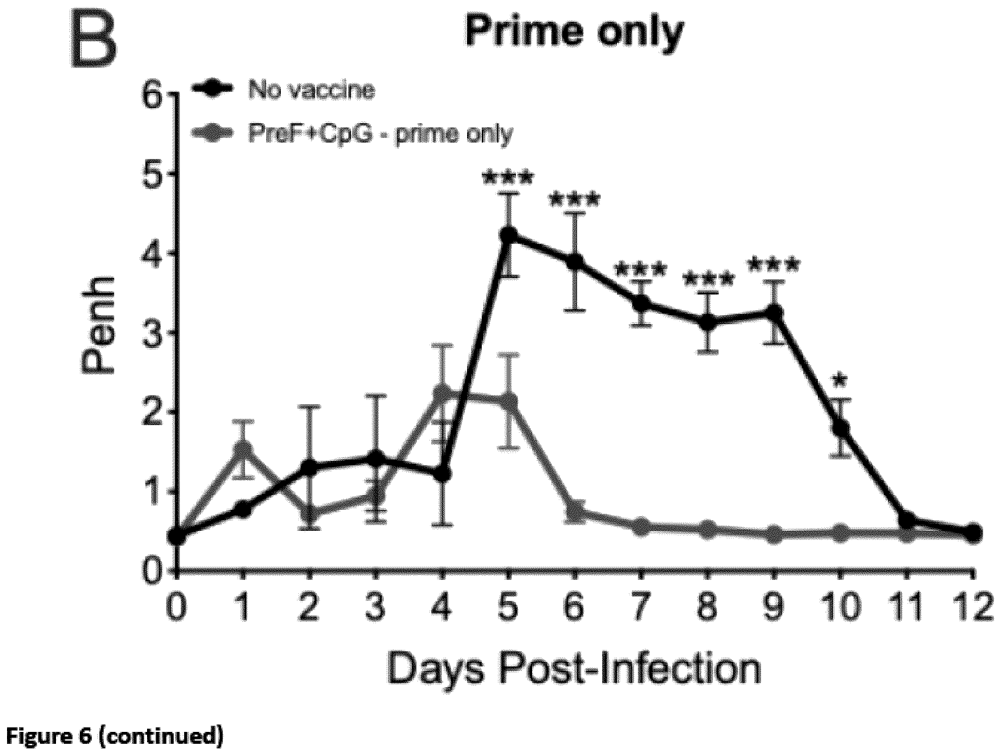

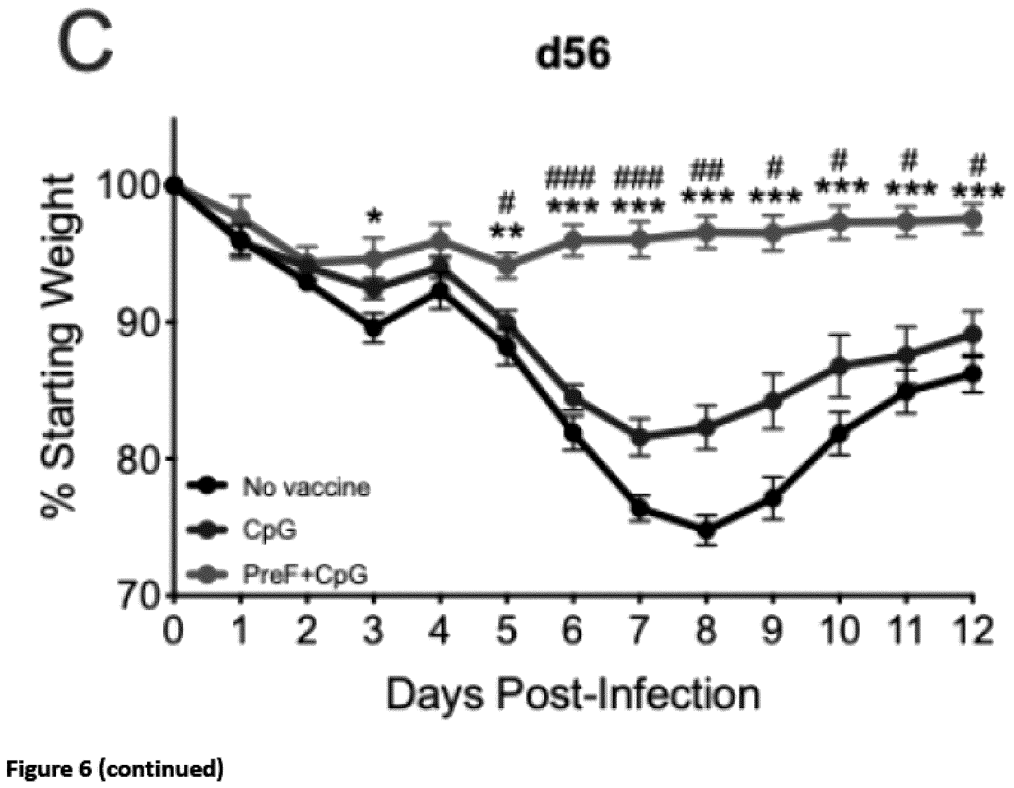

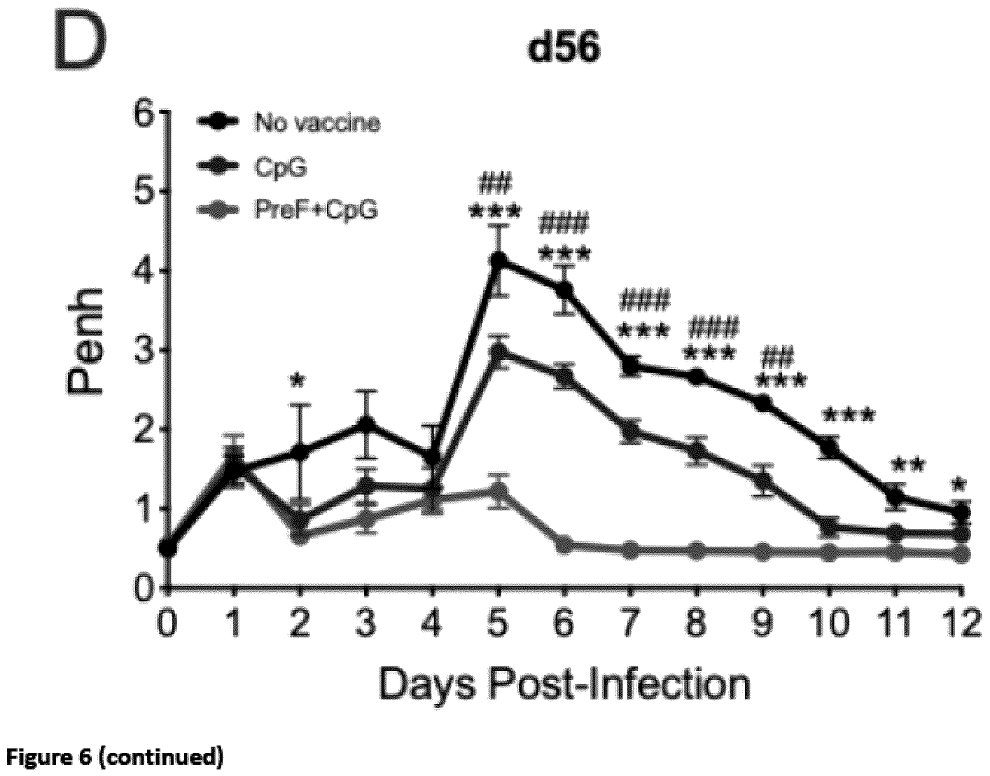

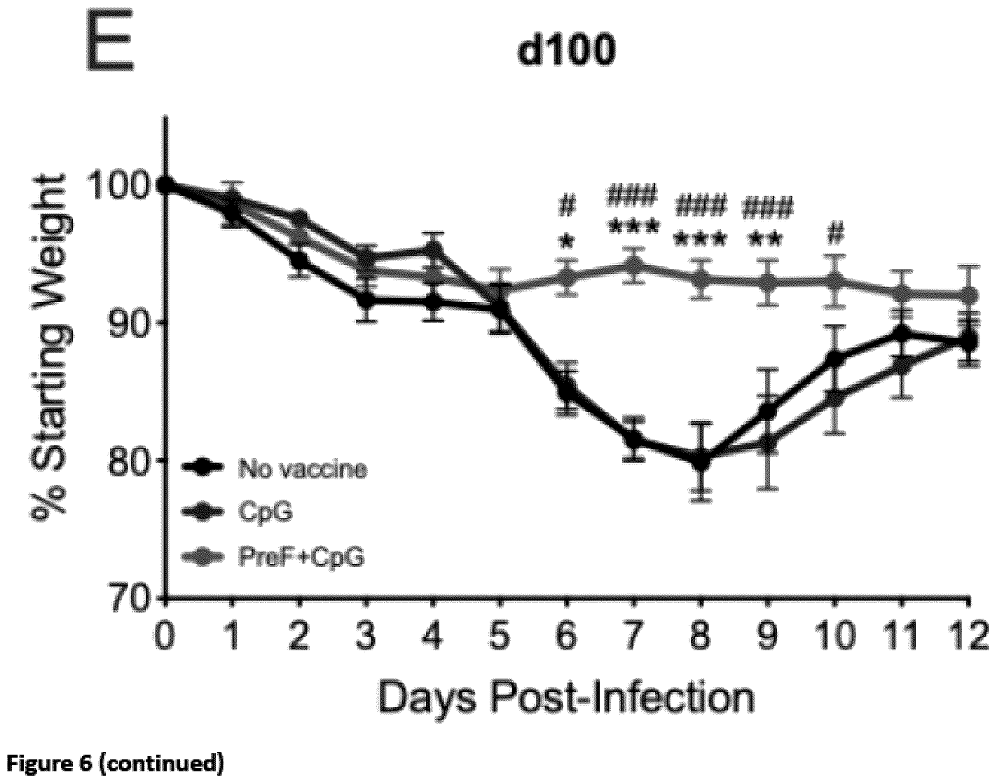

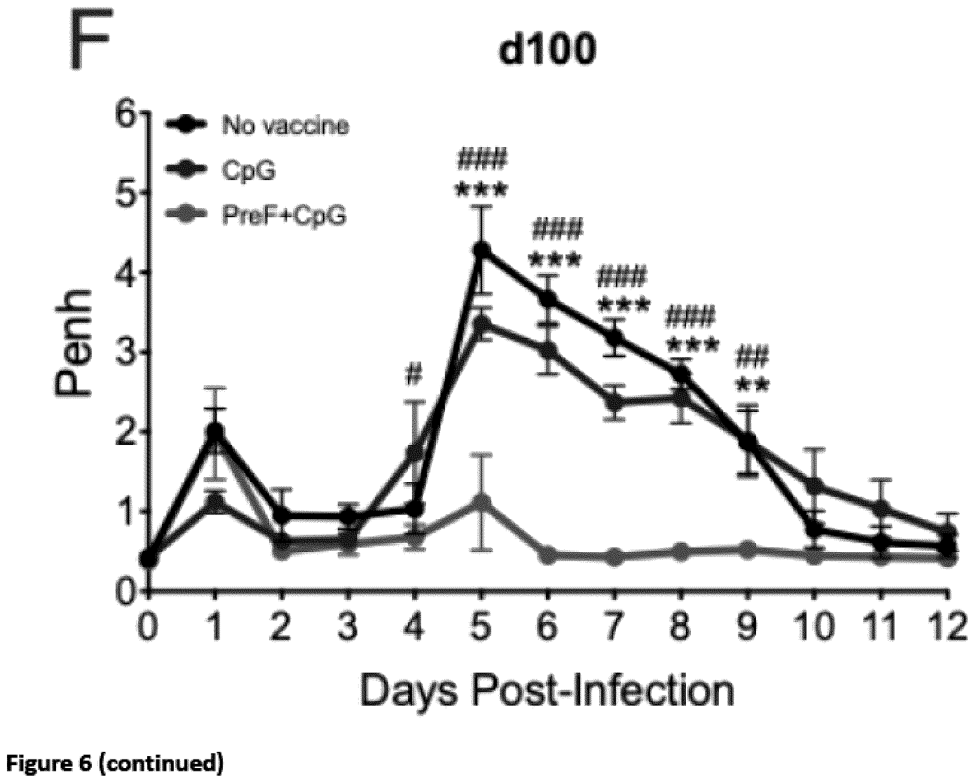

Our data demonstrate that a single prime only immunization with the nanoparticle formulation containing RSV pre-F+CpG adjuvant is sufficient to provide protection against RSV-induced disease including weight loss and airway dysfunction (e.g Penh) as compared to unimmunized control mice or mice immunized with nanoparticles containing the CpG adjuvant only (without the PreF antigen). (See FIG. 6). Prime only immunization is also sufficient to significantly reduce virus replication in the lung as compared to unimmunized mice or mice immunized with nanoparticles containing the CpG adjuvant only (without the PreF antigen). Mice immunized using a prime/boost immunization approach also exhibit significant protection against RSV-induced disease including weight loss and airway dysfunction (e.g Penh) as compared to unimmunized mice when challenged either at 56 days post-prime (see FIGS. 6C and 6D) or Day 100 post-prime (see FIGS. 6E and 6F).

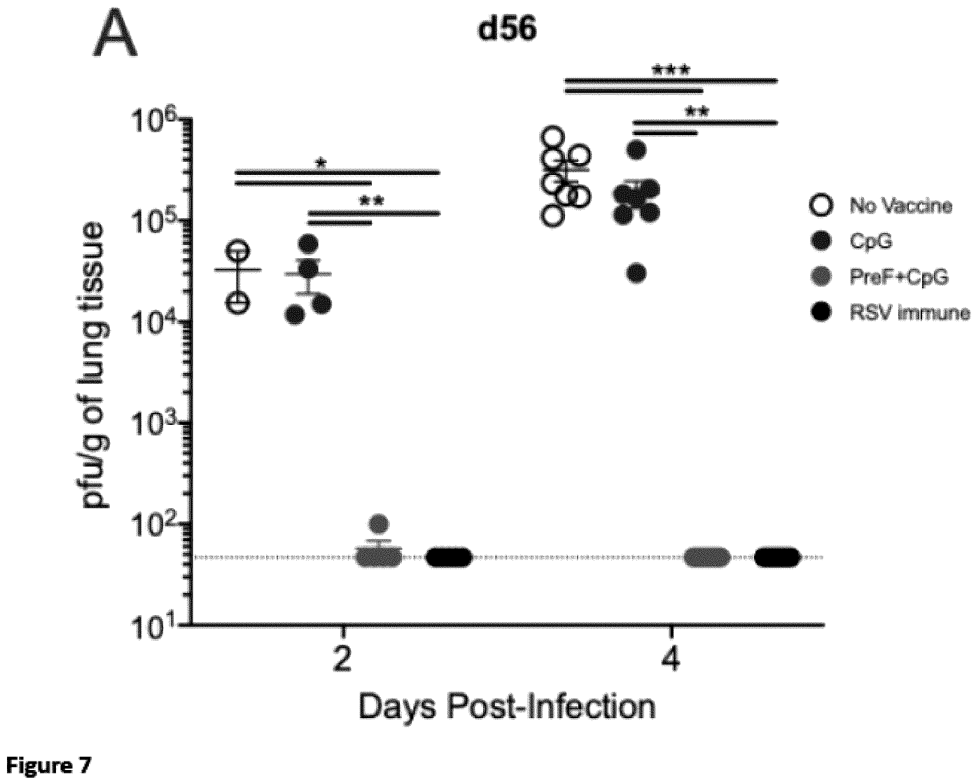

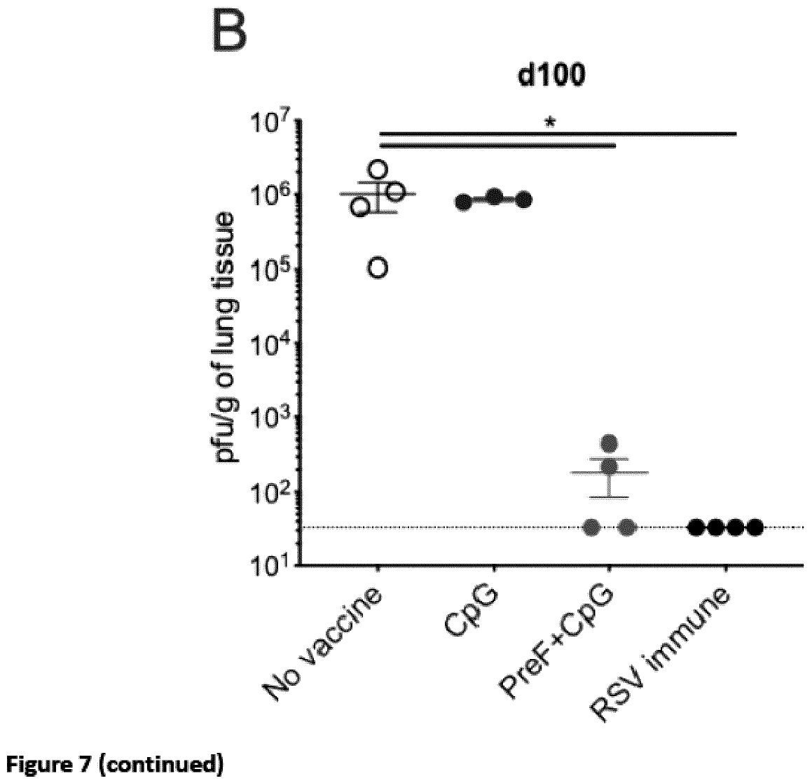

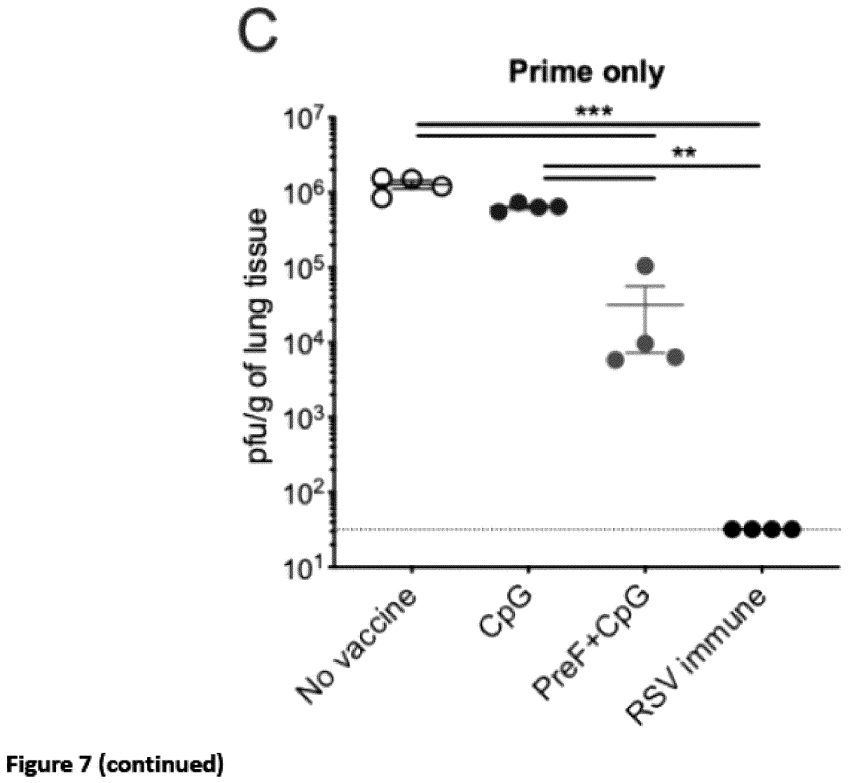

Mice that received a prime/boost immunization exhibited significantly reduced virus replication in the lung as compared to unimmunized mice or mice immunized with nanoparticles containing the CpG adjuvant only (without the PreF antigen) as shown in FIG. 7A (day 56 challenge) or FIG. 7B (day 100 challenge).

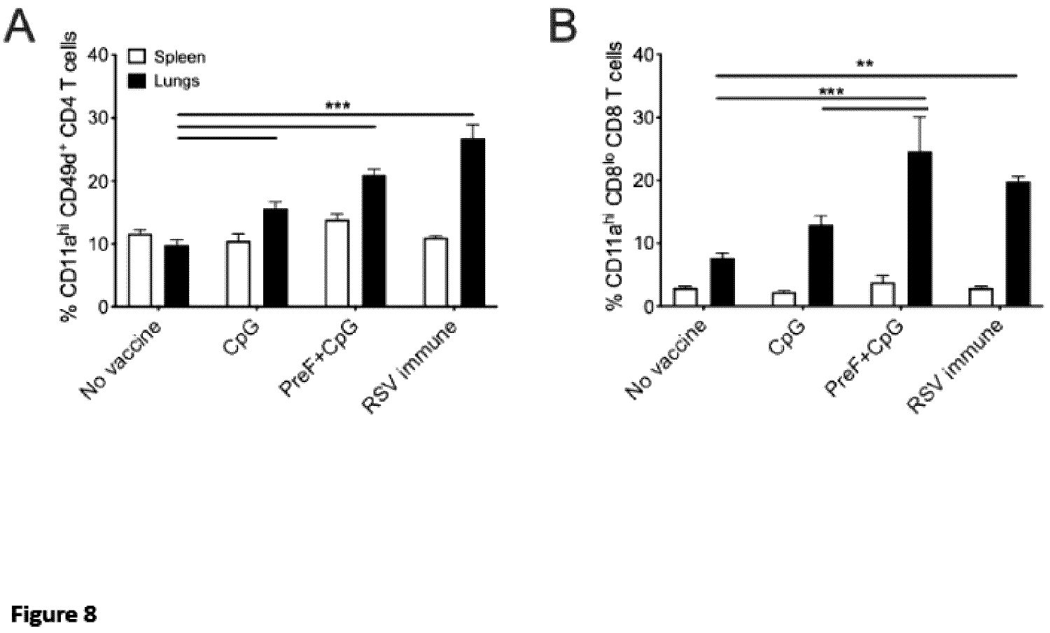

Our data indicate that prime/boost vaccination with PreF+CpG nanoparticles elicits an increase in activated CD4 and CD8 T cells as compared to mice immunized with nanoparticles containing only CpG or unimmunized mice (FIGS. 8A and 8B). This data is consistent with the induction of a T cell response following the nanoparticle vaccination.

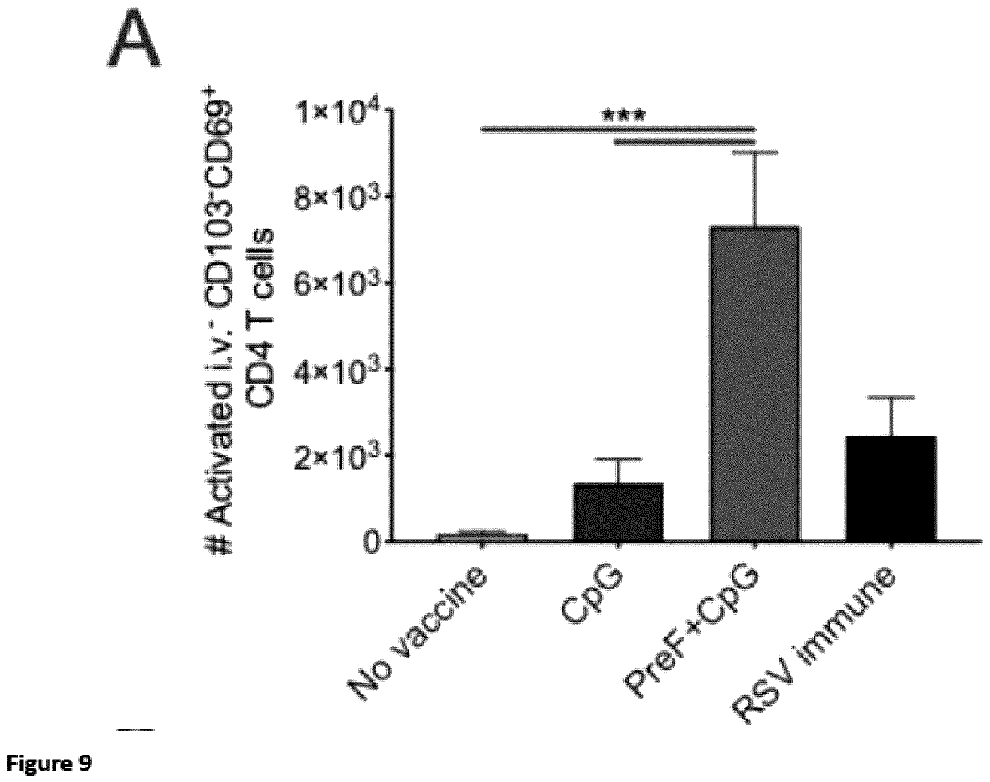

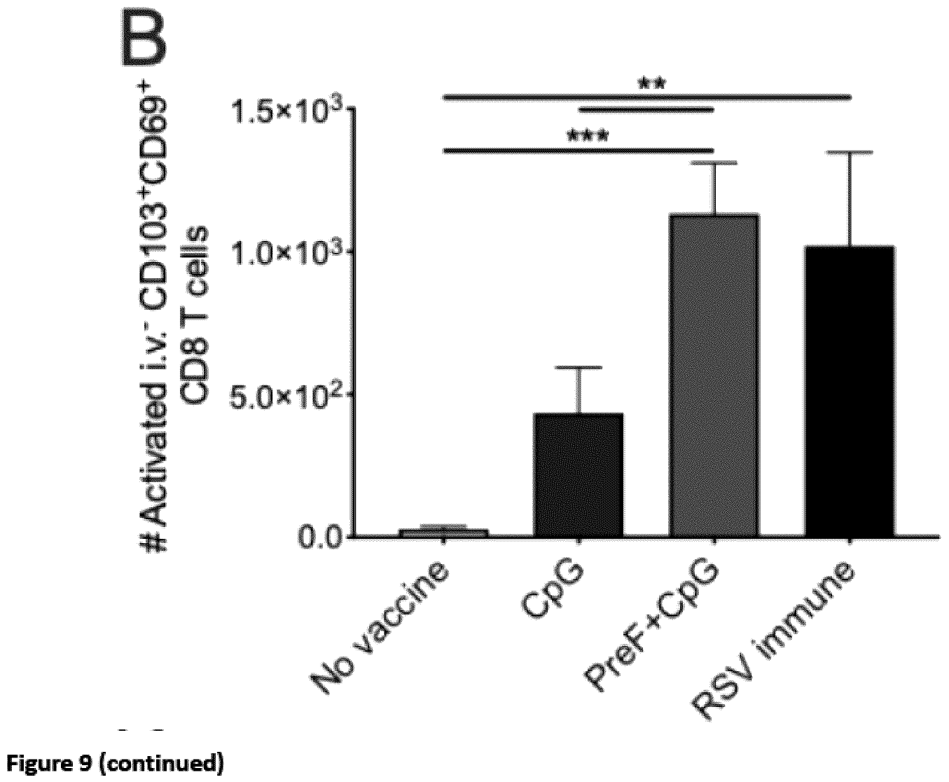

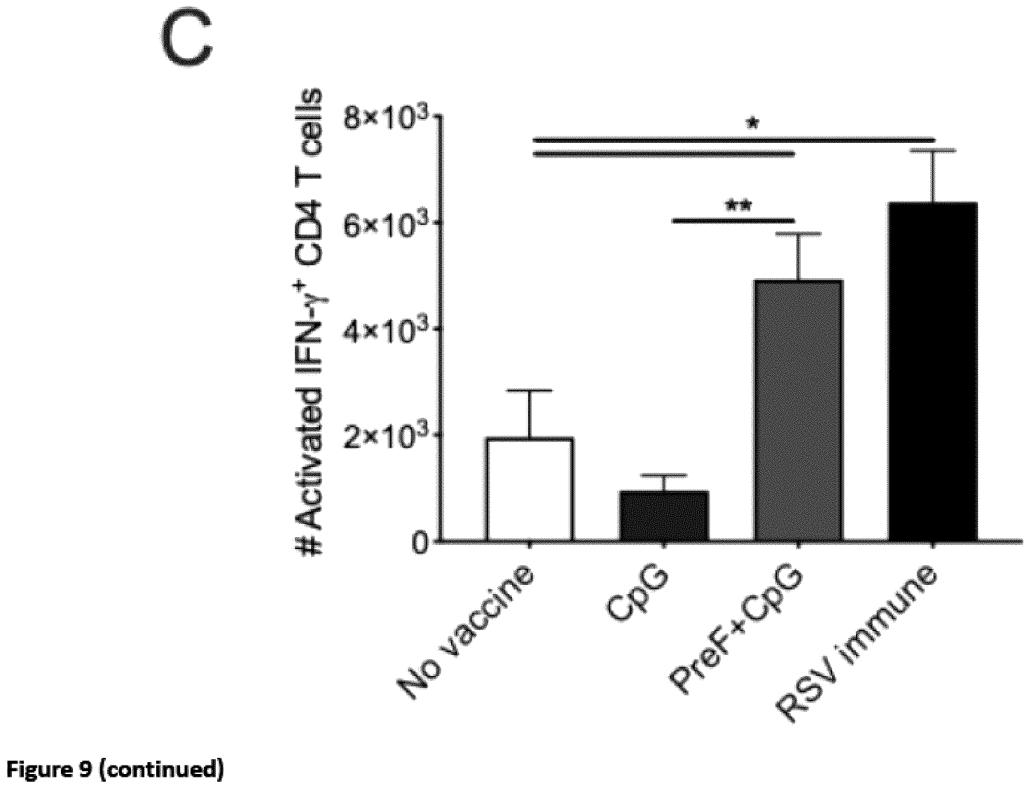

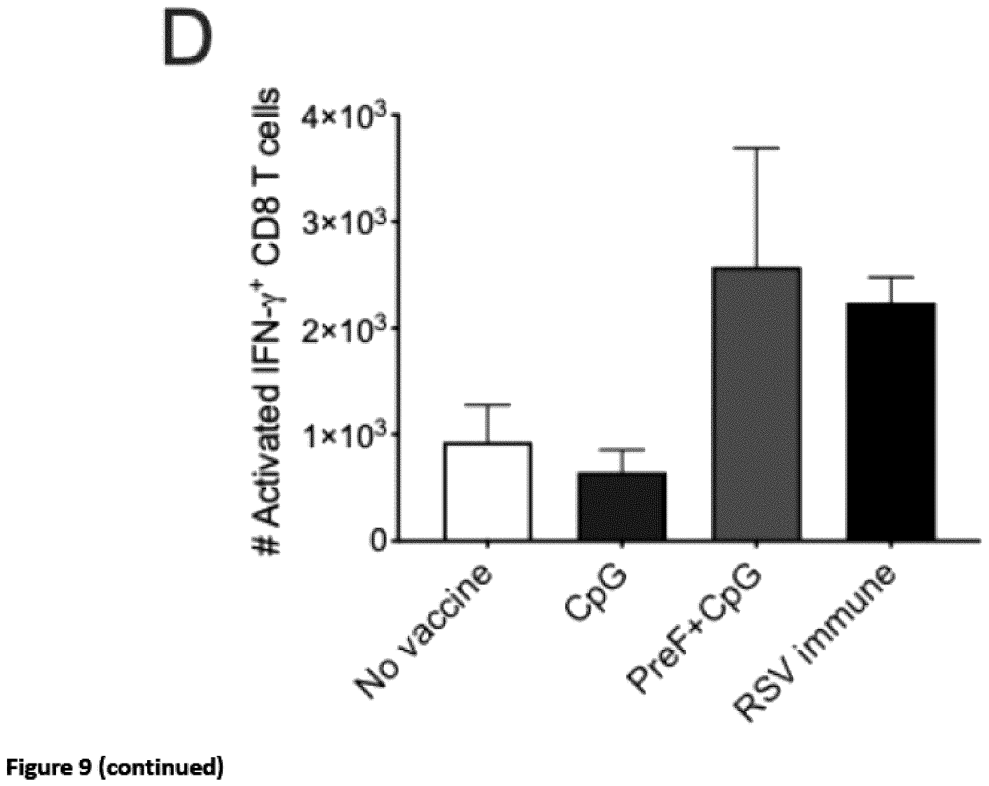

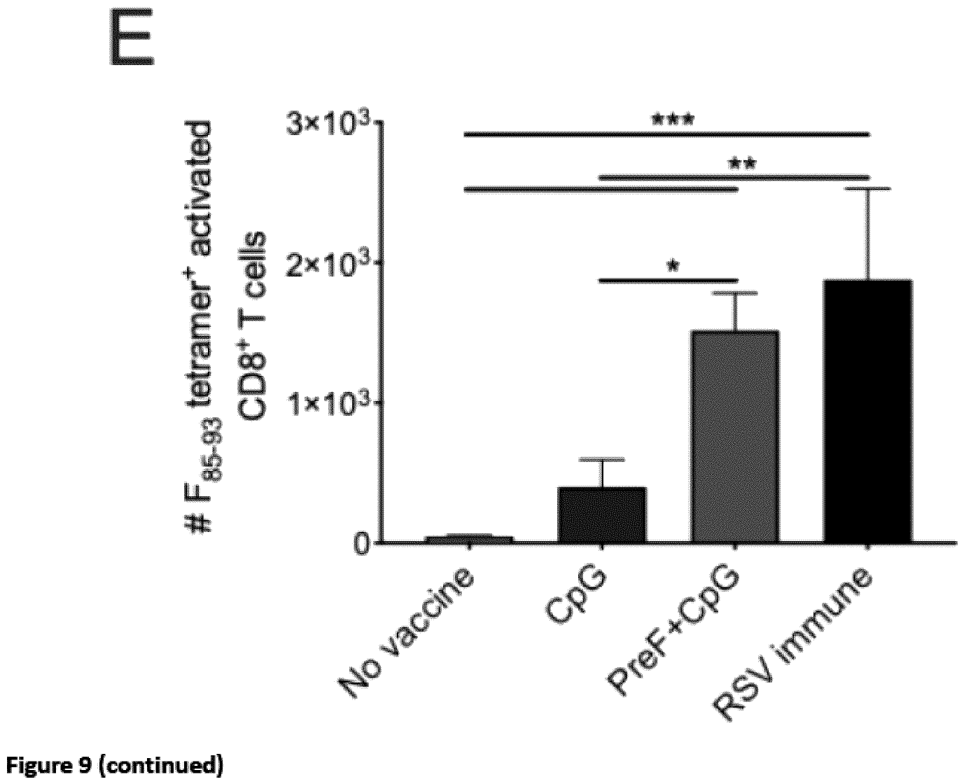

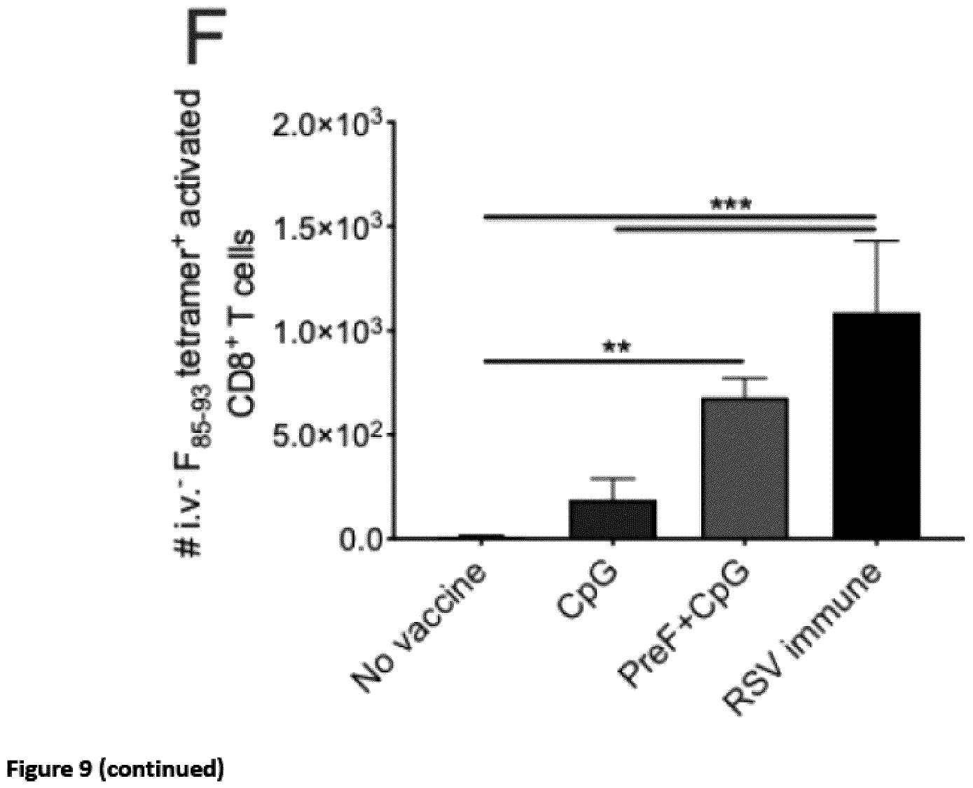

Additional data indicate that the activated CD4 and CD8 T cells exhibit phenotypic cell surface markers (CD69⁺ for CD4 T cells and CD69⁺CD103⁺ for CD8 T cells) that is consistent with a tissue-resident population (e.g. Trm) that may be important in providing protection (FIGS. 9A and 9B). In addition, the activated CD4 and CD8 T cells make IFN-γ following stimulation indicating that they are of a Th1 and Tc1 phenotype, respectively (FIGS. 9C and 9D).

Using MHC-class I tetramers to identify RSV-specific CD8 T cells, we observed a significant increase in the total number of RSV tetramer-specific CD8 T cells specific to the F85-93 CD8 T cell epitope 2 weeks following the boost immunization (eg day 42 post-prime) as compared to unimmunized mice or mice immunized with nanoparticles containing the CpG adjuvant only (without the PreF antigen) as shown in FIG. 9E. FIG. 9F shows that these RSV tetramer-staining cells are located in the lung tissue.

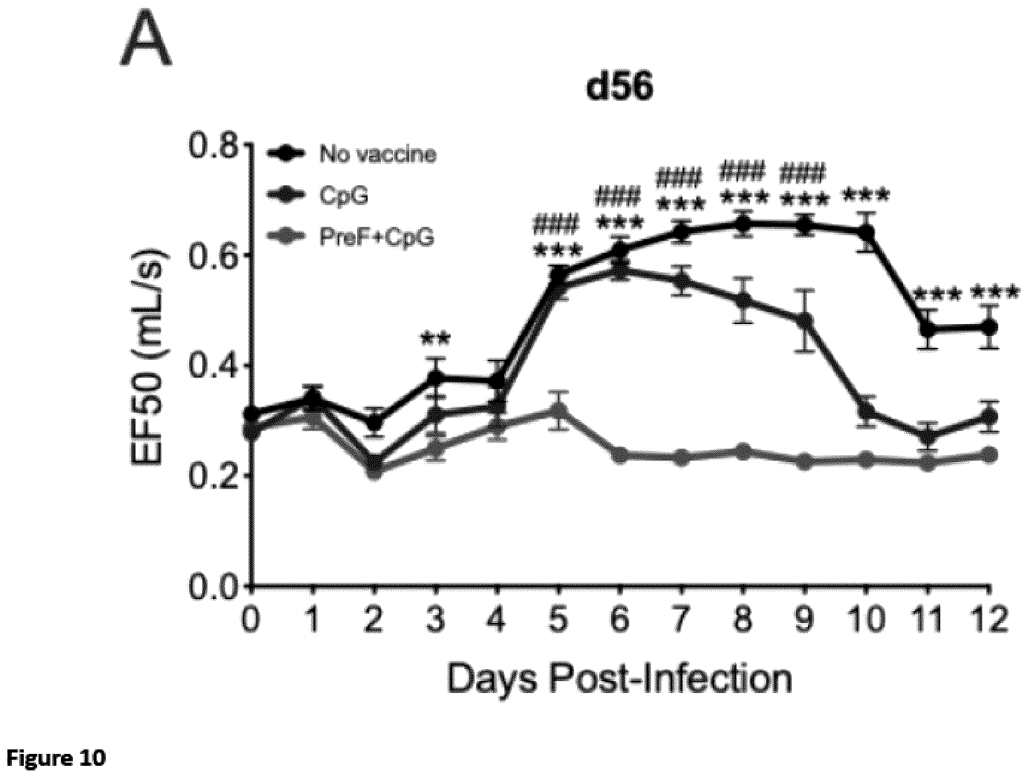

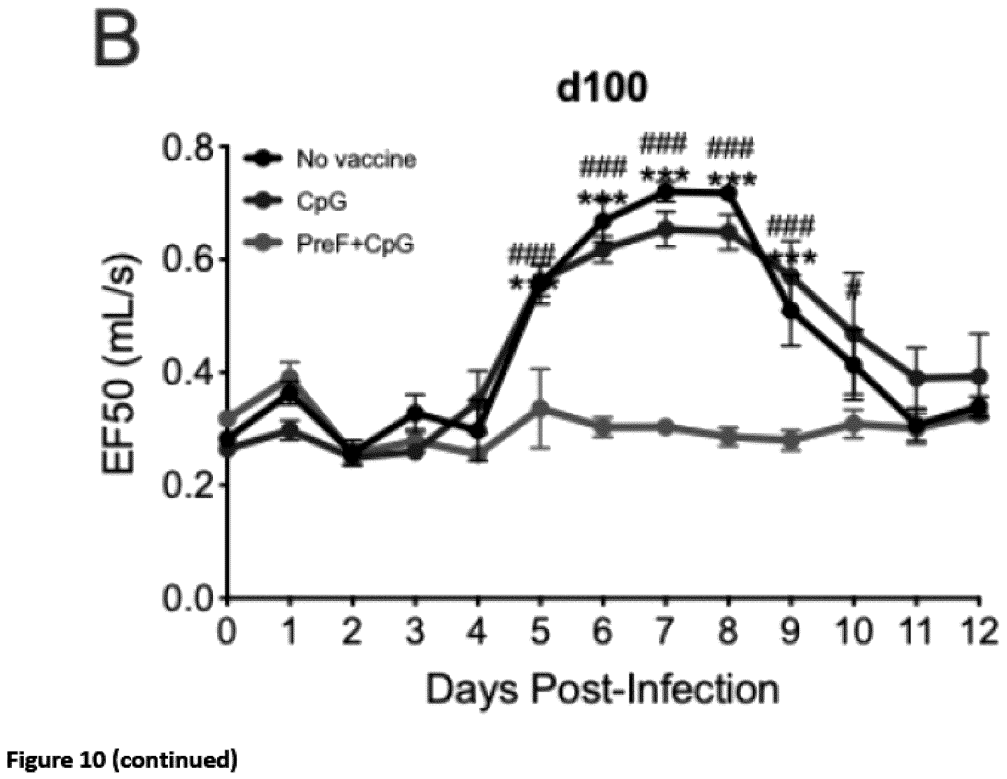

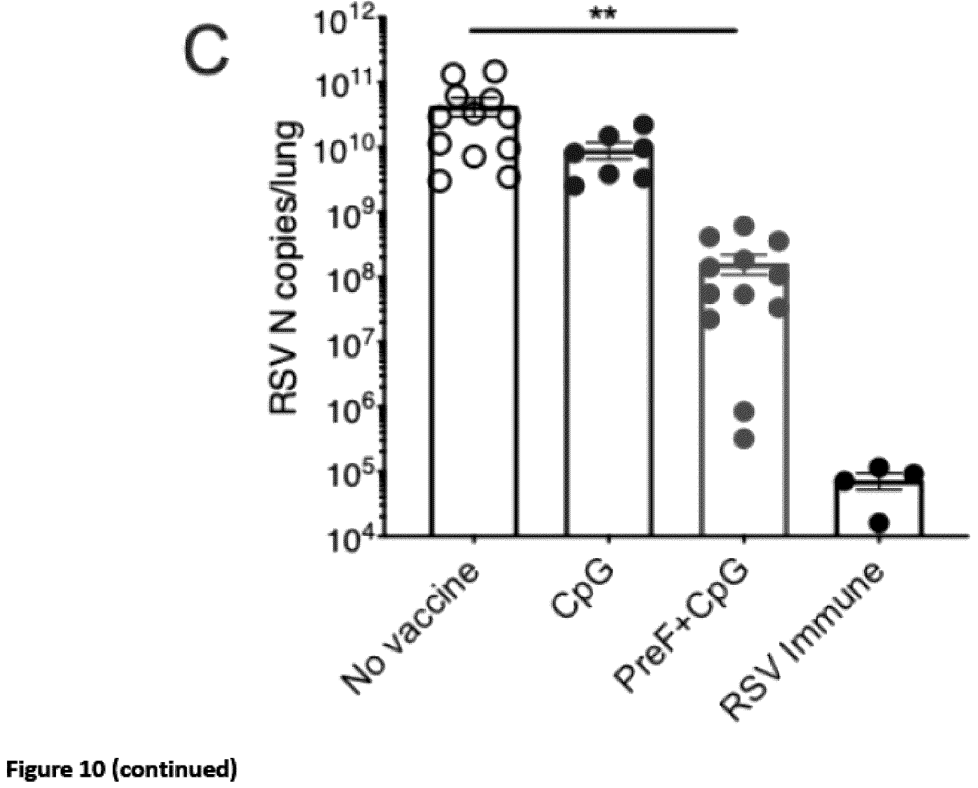

FIG. 10 represents additional data demonstrating that mice immunized using a prime/boost immunization approach exhibit significant protection against RSV-induced airway dysfunction (e.g EF50) as compared to unimmunized mice when challenged either at 56 days post-prime (FIG. 5A) or at 100 days post-prime (FIG. 5B). FIG. 5C demonstrates additional data showing that mice immunized using a prime/boost immunization approach using PreF+CpG nanoparticles exhibit significant protection against RSV replication as measured by RT-PCR for the RSV N gene as compared to mice immunized with nanoparticles containing only CpG or unimmunized mice.

It will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

Citations to a number of patent and non-patent references are made herein. The cited references are incorporated by reference herein in their entireties. In the event that there is an inconsistency between a definition of a term in the specification as compared to a definition of the term in a cited reference, the term should be interpreted based on the definition in the specification.

We claim:

1. A vaccine composition comprising an effective amount of respiratory syncytial virus (RSV) F protein in a pre-fusion stabilized form and/or M protein incorporated into biodegradable polyanhydride polymer particles for inducing an immune response against RSV.

2. The vaccine composition of claim 1, comprising an effective amount of RSV F protein in the pre-fusion stabilized form incorporated into the biodegradable polyanhydride polymer particles for inducing an immune response against RSV.

3. The vaccine composition of claim 1, wherein the F protein in a pre-fusion stabilized form is selected from the group consisting of DS-Cav1, DS-TriC, Vav-1-TriC, DX-Cav1-TriC, Pre-F-GCN4t, SC-DM, and SC-TM.

4. The vaccine composition of claim 1, wherein the F protein in a pre-fusion stabilized form is DS-Cav1.

5. The vaccine composition of claim 1, comprising an effective amount of RSV M protein incorporated into the biodegradable polyanhydride polymer particles for inducing an immune response against RSV.

6. The vaccine composition of claim 1, further comprising an adjuvant.

7. The vaccine composition of claim 6, wherein the adjuvant is incorporated into the biodegradable polyanhydride polymer particles.

8. The vaccine composition of claim 1, further comprising a CpG oligonucleotide.

9. The vaccine composition of claim 8, wherein the CpG oligonucleotide is incorporated into the biodegradable polyanhydride polymer particles.

10. The vaccine composition of claim 8, wherein the CpG oligonucleotide is a CpG oligodeoxynucleotide (ODN).

11. The vaccine composition of claim 10, wherein the CpG oligonucleotide is incorporated into the biodegradable polyanhydride polymer particles.

12. The vaccine composition of claim 1, wherein the biodegradable polyanhydride polymer particles have an average effective diameter of less than about 5 μM.

13. The vaccine composition of claim 1, wherein the vaccine composition induces an antibody response as well as a CD4 T cell response when administered to a subject in need thereof.

14. The vaccine composition of claim 1, wherein the biodegradable polyanhydride polymer particles comprise a polymer formed from a 1,ω-bis(p-carboxyphenoxy)(C₂-C₁₂)alkane, a 1,ω-bis(p-carboxyphenoxy)(C₂-C₁₂)dioxa-alkane, and a (C₅-C₂₀)alkanoic diacid.

15. The vaccine composition of claim 1, wherein the biodegradable polyanhydride polymer particles comprise a polymer formed from 1,6-bis(p-carboxyphenoxy)hexane (CPH), 1,8-bis(p-carboxyphenoxy)-3,6-dioxaoctance (CPTEG), and sebacic acid (SA).

16. The vaccine composition of claim 1, wherein the biodegradable polyanhydride polymer particles comprise a polymer formed from 1,6-bis(p-carboxyphenoxy)hexane (CPH), 1,8-bis(p-carboxyphenoxy)-3,6-dioxaoctance (CPTEG), and sebacic acid (SA), wherein the polymer is formed from CPH and CPTEG at a ratio range of CPH:CPTEG of 80-60:20-40.

17. The vaccine composition of claim 1, further comprising an RSV protein selected from NS1, NS2, N, P, SH, G, M2-1, M2-2, L, or any combinations thereof.

18. A method comprising administering the vaccine of claim 1 to a subject who is at risk for infection by RSV.

19. The method of claim 18, wherein after the vaccine composition is administered to the subject, the subject is protected against infection by RSV.

20. The method of claim 18, wherein the vaccine composition is administered by a route selected from intranasal, pulmonary, oral, subcutaneous, intramuscular, or intravenous.
